(19) United States
(12) Patent Application Publication
(10) Pub. No.: US 2025/0206768 A1
(43) Pub. Date: Jun. 26, 2025

(54) LARGE SCALE PRODUCTION OF N-ACETYLLACTOSAMINE DERIVATIVES

(71) Applicant: KODE BIOTECH LIMITED, Auckland (NZ)

(72) Inventors: Nicolai BOVIN, Moscow (RU); Roman KUNETSKIY, Moscow (RU)

(21) Appl. No.: 18/851,961

(22) PCT Filed: Apr. 6, 2023
(86) PCT No.: PCT/IB2023/053505

§ 371 Date: Sep. 27, 2024

(30) Foreign Application Priority Data
Apr. 8, 2022 (AU) 2022900930

(51) Int. Cl. C07H 5/06

(52) U.S. Cl. CPC C07H 5/06

(57) ABSTRACT

Methods of preparing N-acetyllactosamine derivatives for use in the preparation 3′-O-glycosyl- and 2′,3′-di-O-glycosyl-N-acetyllactosamine derivatives are described. The methods avoid the use of column chromatography and allow for the preparation of multigram quantities of the synthons.

### TECHNICAL FIELD

The invention relates to methods of preparing 3′-OH and 2′,3′-di-OH N-acetyllactosamine derivatives for use in the preparation 3′-O-glycosyl- and 2′,3′-di-O-glycosyl-N-acetyllactosamine derivatives, including glycoconjugates comprising the glycans GalNAcα3Galβ4GlcNAc, Galα3Galβ4GlcNAc (Galili), GalNAcα3(Fucα2)Galβ4GlcNAc (Aₜₑₜᵣₐ) or Galα3(Fucα2)Galβ4GlcNAc (Bₜₑₜᵣₐ). In particular, although not exclusively, the invention relates to a scalable method of preparing ω̅-trifluoroacetamidoalkyl 2-acetamido-3-O-acetyl-6-O-benzyl-2-deoxy-4-O-[2,4,6 tri-O-acetyl-β-D-galactopyranosyl]-β-D-glucopyranoside for use in the preparation of 3′-O-glycosyl-N-acetyllactosamine conjugates, such as the clinical candidate AGI-134 (CAS RN 1821461-84-0).

### BACKGROUND ART

The structure of the glycan component of glycolipids and glycoproteins is often the determinant of the biological function of these glycoconjugates. Interest in the preparation of oligosaccharides of a predetermined structure according to scalable methods arises from the potential diagnostic, prognostic and therapeutic uses of these determinants of biological function (glycotopes). Many oligosaccharides with potential use in these areas are glycosylated derivatives of the disaccharide N-acetyllactosamine (Galβ4GlcNAc).

The publication of Hindsgaul et al (1982) discloses the synthesis of blood group antigenic determinants derived from the disaccharide N-acetyllactosamine. The blood group antigen determinants include 2′-O-glycosylated derivatives of N-acetyllactosamine such as the trisaccharides Fucα2Galβ4GlcNAc (H type 2) and Fucα2Galβ4(Fucα3)GlcNAc (Y). The derivatives were prepared as 8-carboxymethyloctanol glycosides. Glycosyl donors were prepared from 3,4,6-tri-O-benzyl-1,2-orthoesters prepared according to the methods disclosed in the publication Borën et al (1973). Glycosyl acceptors having an available 4-OH group were prepared from 8-methoxycarbonyloctyl 2-acetamido-4,6-O-benzylidene-2-deoxy-β-D-glucopyranoside prepared according to the methods disclosed in the publication of Lemieux et al (1979). Selective glycosylation of the 4-hydroxyl group of the N-acetylglucosamine derivative was achieved by first treating 8-methoxycarbonyloctyl 2-acetamido-4,6-O-benzylidene-2-deoxy-β-D-glucopyranoside with benzyloxymethyl halide to form 8-methoxycarbonyloctyl-2-acetamido-3-O-benzyloxymethyl-4,6-O-benzylidene-2-deoxy-β-D-glucopyranoside, the 3-O-benzyloxymethyl group of which proved sufficiently stable to acidic hydrolysis to allow removal of the 4,6-O-benzylidene group by 50% aqueous acetic acid at 80° C. The 6-hydroxyl group of the 4,6-diol product was then preferentially alkylated using benzyloxymethyl halide to yield an otherwise protected 8-methoxycarbonyloctyl 2-acetamido-3,6-di-O-benzyloxymethyl-2-deoxy-β-D-glucopyranoside having the required 4-hydroxyl group. Condensation of this glycosyl acceptor with a p-nitrobenzoate-bromide glycosyl donor yielded a mixture of α- and β-linked isomers that were then readily separated in their N-acetylated form following removal of the p-nitrobenzoyl group to provide the N-acetyllactosamine derivative 8-methoxycarbonyloctyl 2-acetamido-3,6-di-O-benzyloxymethyl-2-deoxy-4-O-(3,4,6-tri-O-benzyl-β-D-galactopyranosyl)-β-D-glucopyranoside.

The publication of Paulsen (1985) provides a review of the methods then available for the preparation of oligosaccharides comprising specific glycosidic linkages. According to the “neighbouring group assisted procedure” an acylated α-D-glycosyl halide is used as the glycosyl donor. The glycosidic linkage is formed via a dioxocarbonium ion intermediate. A β-D-galactopyranoside may be prepared according to this procedure. According to the “in situ anomerisation procedure” an α-D-pyranosyl halide with a non-neighbouring group active constituent at C-2 is used. The presence of a catalyst establishes an equilibrium between the α-D-pyranosyl and β-D-pyranosyl halides, the lesser stability of the latter favouring the formation of the α-D-glycopyranoside. The use of thioglycosides according to the methods described in the publication of Lönn (1984) is also briefly reviewed.

The publication of Hanessian et al (2001a) discloses the development of synthetic protocols that avoid the use of thiol-based reagents, hydrolytically labile glycosyl donors, and potentially toxic reagents. The objective was to develop protocols adaptable for the production of relatively large quantities of Galili triose antigen in a process group environment. The protocols developed utilise partially protected and unprotected 3-methoxy-2-pyridyl (MOP) glycosides as acceptors and donors in the production of the target triose. The preparation of a lactosamine acceptor utilizing a substituted MOP Galβ1-4 donor and an MOP GlcNAc acceptor is considered. The reaction between 3-benzyloxycarbonylamino-1-propyl 6-O-tert-butyldiphenylsily-2-deoxy-2-phthalimido-β-D-glucopyranoside as acceptor and 3-methoxy-2-pyridyl 2,3,4-tri-O-benzoyl-6-O-tert-butyldiphenylsilyl-β-D-galactopyranoside as donor yielded a mixture of 3-O and 4-O monosubstituted disaccharide with the 4-O monosubstituted disaccharide as the major component. An approach to the production of the target triose identified as “more practical” and utilising an alkyl N-acetyl lactosaminide available via a chemoenzymatic route was also explored.

The publication of Pazynina et al (2002) discloses the α-fucosylation of an aminoalkyl derivative of N-acetyllactosamine having a pair of free hydroxyl groups at C-2′ and C-3 and the α-galactosylation of an aminoalkyl derivative of N-acetyllactosamine having a pair of free hydroxyl groups at C-3′ and C-4′. The latter diol was obtained as an aminoalkyl derivative by the sequential O-acetylation and removal of the isopropylidene protection of the N-acetyllactosamine derivative formed from the glycosylation of the acceptor (3-trifluoroacetamidopropyl)-2-acetamido-3-O-acetyl-6-O-benzyl-2-deoxy-β-D-glucpyranoside with the donor 2,3,4-tri-O-acetyl-6-O-benzyl-β-D-galactopyranosyl bromide followed by the Zemplen deacetylation and acetonation at the 3′,4′-positions.

The publication of Severov et al (2007) discloses the preparation of several N-acetyllactosamine derivatives substituted at either or both of the 3′-OH and 6′-OH positions. A 2-azidoethyl spacered N-acetyllactosamine was used as the starting compound for the preparation of the substituted N-acetyllactosamine derivatives. Hydrogenation of the azide group resulted in an amine, which was protected with a N-trifluoroacetyl group by treatment with methyl trifluoroacetate in methanol. The substitution of 2-trifluoroacetamidoethyl spacer for the azidoethyl moiety was executed at an initial step of the synthesis, because the 2-trifluoroacetamidoethyl protective group was stable in the further synthetic procedures (in particularly hydrogenolysis) and could be quantitatively removed at the final step by an aqueous base. Of the two possible strategies for the creation of branching at the galactose residue, glycosylation at position 3 followed by glycosylation at position 6 of the galactose residue was chosen. This was because the primary hydroxyl at position 6 was more reactive than the secondary hydroxyl, and the effective glycosylation of the former was still possible following the introduction of a bulky substituent at position 3. The benzylidene protective group was introduced into 2-trifluoroacetamidoethyl 2-acetamido-2-deoxy-4-O-(β-D-galactopyranosyl)-β-D-glucopyranoside (2-trifluoroacetamidoethyl N-acetyllactosamine) by treatment with dimethoxytoluene in the presence of p-toluenesulfonic acid. The resulting benzylidene derivative was acetylated with acetic anhydride in pyridine without additional purification. The total yield of 2-trifluoroacetamidoethyl 2-acetamido-3,6-di-O-acetyl-2-deoxy-4-O-(2,3,4-tri-O-acetyl-6-O-benzyl-β-D-galactopyranosyl)-β-D-glucopyranoside after these two steps is reported as 87%. The 2-trifluoroacetamidoethyl 2-acetamido-3,6-di-O-acetyl-2-deoxy-4-O-(2,3,4-tri-O-acetyl-6-O-benzyl-β-D-galactopyranosyl)-β-D-glucopyranoside was then deacetylated by Zemplen in 86% yield. Introduction of the 3′,4′-orthoester protective group (Mukhopadhyay and Field (2003)) with triethyl orthoacetate in the presence of p-toluenesulfonic acid in acetonitrile followed by acetylation and opening of the orthoester cycle with acetic acid permitted the obtaining of 2-trifluoroacetamidoethyl 2-acetamido-3,6-di-O-acetyl-2-deoxy-4-O-(2,4-di-O-acetyl-6-O-benzyl-β-D-galactopyranosyl)-β-D-glucopyranoside in 70% yield. (A lactosamine derivative with the free OH group in position 4 of galactose and acetyl group at C3 was apparently generated upon the chromatography on silica gel due to migration of the acetyl group. This was the main by-product of the transformation and the proportion of this 4-OH derivative could be decreased by the addition of 0.5% of pyridine in the eluent for chromatography.)

The publication of Pazynina et al (2008) also discloses the preparation of N-acetyllactosamine derivatives Galβ1-4GlcNAcβ-sp, where ‘sp’ is a primary amino alkyl spacer, e.g., aminopropyl (—O(CH₂)₃NH₂). Preparation was via the glycosylation of the glucosamine derivative 3-trifluoroacetamidopropyl 2-acetamido-3-O-acetyl-6-O-benzyl-2-deoxy-4-O-β-D-glucopyranoside with the glycosyl bromide 2,3,4-O-triacetyl-6-O-benzyl-α-D-galactopyranosyl bromide (obtained from the corresponding thioglycoside) at a molar ratio of 1:2. This provided after 2-fold purification by chromatography a 61% yield of the blocked N-acetyllactosamine derivative 3-trifluoroacetamidopropyl 2-acetamido-3-O-acetyl-6-O-benzyl-2-deoxy-4-O-(2,3,4-tri-O-acetyl-6-O-benzyl-β-D-galactopyranosyl)-β-D-glucopyranoside. The disaccharide obtained by Zemplen deacetylation of this derivative was acetylated via an intermediate orthoacetate and provided following chromatography a 59% yield of 3-trifluoroacetamidopropyl 2-acetamido-3-O-acetyl-6-O-benzyl-2-deoxy-4-O-(2,4-di-O-acetyl-6-O-benzyl-β-D-galactopyranosyl)-β-D-glucopyranoside. This otherwise blocked 3′-OH N-acetyllactosamine derivative was then used in the preparation of 3-trifluoroacetamidopropyl 2-acetamido-3-O-acetyl-6-O-benzyl-2-deoxy-4-O-[2,4-di-O-acetyl-6-O-benzyl-3-O-(2-acetamido-3,4,6-tri-O-acetyl-2-deoxy)-β-D-glucopyranosyl)-β-D-galactopryanosyl]-β-D-glucopyranoside by glycosylation of the 3′-OH N-acetyllactosamine derivative acceptor with [the glycosyl bromide 2-deoxy-2-[[(2,2,2-trichloroethoxy)carbonyl]amino]-3,4,6-O-triacetyl-α-D-glucopyranosyl bromide (obtained from the corresponding thioglycoside).

The publication of Romano and Oscarson (2019) discloses the synthesis of several N-acetyllactosamine derivatives for use in the preparation of oligomers and polymers of N-acetyllactosamine ([Galβ1-4GlcNAc 1-3]mer) starting from lactosamine hydrochloride. Suitable disaccharide blocks are formed and then coupled to obtain oligo-N-acetyllactosamine chains. The synthesis of the 3′-OH N-acetyllactosamine derivative 2-azidoethyl 2-acetamido-3,6-di-O-acetyl-4-O-(2,4,6-tri-O-acetyl-β-D-galactopyranosyl)-2-deoxy-β-D-glucopyranoside is disclosed. Briefly, lactosamine hydrochloride was peracetylated and converted into its oxazoline derivative prior to the introduction of the 2-azidoethyl linker. Deacetylation and regioselective introduction of 3′,4′-O-isopropylidene, followed by acetylation and then cleavage of the isopropylidene ring afforded the corresponding diol. The 3′-OH N-acetyllactosamine derivative was obtained by subsequent 3′,4′-O-orthoester formation and rearrangement in acidic conditions. Save for the unblocked 2-azidoethyl 2-acetamido-4-O-(β-D-galactopyranosyl)-2-deoxy-β-D-glucopryanoside, each of the intermediates in the synthesis of the 3′-OH N-acetyllactosamine derivative from the oxazoline disclosed in the publication of André et al (2010), was purified by flash column chromatography.

The publication of Gagarinov et al (2019) discloses the preparation of an azide analogue of an otherwise blocked 3′-OH derivative of N-acetyllactosamine, i.e., dimethylthexylsilyl 2-azido-2-deoxy-3,6-di-O-benzyl-4-O-(2-O-levulinoyl-4,6-di-O-benzyl-β-D-galactopyranosyl)-β-D-glucopyranoside. The precursor disaccharide dimethylthexylsilyl 2-azido-2-deoxy-3,6-di-O-benzyl-4-O-(2-O-levulinoyl-3-O-(2-naphthyl)-4,6-di-O-benzyl-β-D-galactopyranosyl)-β-D-glucopyranoside is obtained following chromatography of the product of the glycosylation of dimethylthexylsilyl 2-azido-2-deoxy-3,6-di-O-benzyl-β-D-glucopyranose. The 3′-OH of the precursor disaccharide is then deprotected by selective removal of the naphthyl group and the azide analogue purified by chromatography.

The publication of Ryzhov et al (2019) discloses the synthesis of an N-acetyllactosamine based branched oligosaccharide comprising the simultaneous glycosylation of the 3′- and 6′-OH groups of N-acetyllactosamine. The required diol acceptor is prepared via an otherwise blocked 3′-OH derivative of N-acetyllactosamine. This intermediate 3-trifluoroacetamidopropyl 2-acetamido-3,6-di-O-acetyl-2-deoxy-4-O-(2,4-di-O-acetyl-6-O-benzyl-β-D-galactopyranosyl)-β-D-glucopyranoside is prepared by first completely deblocking the spacer-armed N-acetyllactosamine prepared according to the publication of Pazynina et al (2002). Benzylidenation followed by acetylation and reductive opening of the benzylidene acetal moiety of the resulting disaccharide followed by acetylation provides the fully blocked immediate precursor 3-trifluoroacetamidopropyl 2-acetamido-3,6-di-O-acetyl-2-deoxy-4-O-(2,3,4-tri-O-acetyl-6-O-benzyl-β-D-galactopyranosyl)-β-D-glucopyranoside of the intermediate. The 3′-OH group of this precursor is then selectively deblocked via a cyclic 3′,4-orthoester to provide the target intermediate.

The publication of Kunetzky et al (2020) discloses the preparation of both the blood group A and B (type 2) tetrasaccharides and the fucose-free trisaccharides Galα3Galβ4GlcNAc (Galili) and GalNAcα3Galβ4GlcNAc. According to the schemes disclosed, the 3′-OH Galβ4GlcNAc acceptor is glycosylated at room temperature with a glycosyl bromide donor obtained from the corresponding ethyl thioglycoside with AgOTf as promoter. The lactosamine acceptor 3-trifluoroacetamidopropyl 2-acetamido-2-deoxy-3-O-acetyl-4-O-(2,4,6-tri-O-acetyl-β-D-galactopyranosyl)-6-O-benzyl-β-D-glucopyranoside is identified as being prepared according to the methods disclosed in the publication of Pazynina et al (2008).

It is an object of the present invention to provide a scalable method of preparing N-acetyllactosamine acceptors for use in the preparation of therapeutically significant glycotopes. It is an object of the present invention to provide an improved method of preparing therapeutic glycoconjugates. These objects are each to be read in the alternative with the object to provide at least a useful choice.

### SUMMARY OF INVENTION

In a first aspect an N-acetyllactosamine derivative (ω-trifluoroacetamidoalkyl 2-acetamido-6-O-benzyl-2-deoxy-4-O-[β-D-galactopyranosyl]-β-D-glucopyranoside (A)) of the following structure is provided:

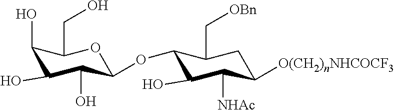

where n is the integer 2, 3, 4, 5, 6, 7 or 8.

Preferably the ω-trifluoroacetamidoalkyl moiety of the N-acetyllactosamine derivative (A) is an ω-trifluoroacetamido-C₂₋₄-alkyl moiety. More preferably the ω-trifluoroacetamidoalkyl moiety is a 3-trifluoroacetamidopropyl moiety.

The N-acetyllactosamine derivative (A) is prepared by the deacetylation of an anomeric mixture of a ω-trifluoroacetamidoalkyl disaccharide (B) of the following structure:

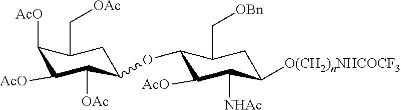

The N-acetyllactosamine derivative (A) is used in the preparation of the W-trifluoroacetamidoalkyl disaccharide (C) of the following structure:

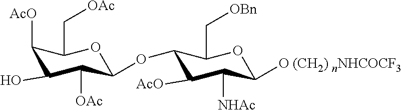

Advantageously, the preparation of the ω-trifluoroacetamidoalkyl disaccharide (C) from the N-acetyllactosamine derivative (A) avoids the use of column chromatography, each of the intermediates being isolatable at sufficient purity in gram (g) quantities by precipitation. The N-acetyllactosamine derivative (A), the ω-trifluoroacetamidoalkyl disaccharide (C), and the intermediates, may therefore each be prepared in batch quantities of greater than 10 g, preferably greater than 30 g.

The ω-trifluoroacetamidoalkyl disaccharide (C) is used in the preparation of ω-trifluoroacetamidoalkyl glycosides (D) of the following structure:

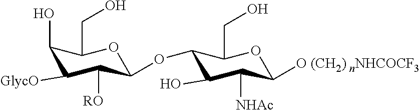

where R is H or Glyc and Glyc is a glycan. Preferably Glyc is a monosaccharide residue. More preferably R is H and Glyc is the monosaccharide residue α-D-galactopyranosyl.

The ω-trifluoroacetamidoalkyl glycosides (D) can be quantitatively converted to the corresponding w-aminoalkyl glycosides (E) by an aqueous base. Examples of ω-aminoalkyl glycosides are Galα3Galβ4GlcNAcβ-O(CH₂)ₙNH₂ (Bₜᵣᵢ(type 2); Galili), GalNAcα3(Fucα2)Galβ4GlcNAcβ-O(CH₂)ₙNH₂ (A (type 2)), Galα3(Fucα2)Galβ4GlcNAcβ-O(CH₂)ₙNH₂ (B (type 2)), Fucα2Galβ4GlcNAcβ-O(CH₂)ₙNH₂ (H (type 2)), Galβ4(Fucα3)GlcNAcβ-O(CH₂)ₙNH₂ (Le^{x}), Fucα2Galβ4(Fucα3)GlcNAcβ-O(CH₂)ₙNH₂ (Le^{y}), and Galα4Galβ4GlcNAcβ-O(CH₂)ₙNH₂ (P₁) In an embodiment of the first aspect an N-acetyllactosamine derivative (3-trifluoroacetamidopropyl 2-acetamido-6-O-benzyl-2-deoxy-4-O-[β-D-galactopyranosyl]-β-D-glucopyranoside (11)) of the following structure is provided:

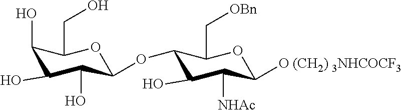

In a second aspect a method for the batch preparation of a quantity of an N-acetyllactosamine derivative (ω-trifluoroacetamidoalkyl 2-acetamido-6-O-benzyl-2-deoxy-4-O-[β-D-galactopyranosyl]-β-D-glucopyranoside (A)) of the following structure is provided:

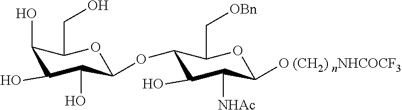

where n is the integer 2, 3, 4, 5, 6, 7 or 8. Preferably the quantity is greater than 10 g. More preferably the quantity is greater than 30 g.

The method comprises the crystallisation of the N-acetyllactosamine derivative (A) from a solution obtained via the deacetylation of an anomeric mixture of a ω-trifluoroacetamidoalkyl disaccharide (B) of the following structure:

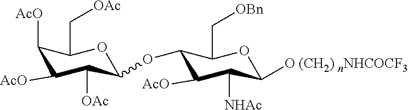

Advantageously the crystallisation provides for the resolution of the deacetylated anomers. Preferably the crystallisation of the N-acetyllactosamine derivative (A) is from a solution in greater than 98% (v/v) acetonitrile.

Preferably the ω-trifluoroacetamidoalkyl moiety of the N-acetyllactosamine derivative (A) and the anomers of the ω-trifluoroacetamidoalkyl disaccharide (B) is a ω-trifluoroacetamido-C₂₋₄-alkyl moiety. More preferably the ω-trifluoroacetamidoalkyl moiety is a 3-trifluoroacetamidopropyl moiety.

In a third aspect a batch quantity of crystalline N-acetyllactosamine derivative (ω-trifluoroacetamidoalkyl 2-acetamido-6-O-benzyl-2-deoxy-4-O-[β-D-galactopyranosyl]-β-D-glucopyranoside (A)) where the quantity is greater than 10 g is provided. More preferably the quantity is greater than 30 g.

Preferably the ω-trifluoroacetamidoalkyl moiety of the crystalline N-acetyllactosamine derivative (A) is a ω-trifluoroacetamido-C₂₋₄-alkyl moiety.

More preferably the ω-trifluoroacetamidoalkyl moiety is a 3-trifluoroacetamidopropyl moiety.

In a fourth aspect a method for the batch preparation of a quantity of at least 90% (w/w) ω-trifluoroacetamidoalkyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (F) of the following structure is provided:

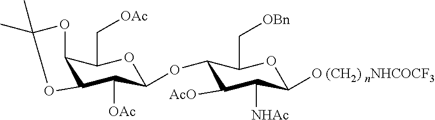

where n is the integer 2, 3, 4, 5, 6, 7 or 8.

The method comprises:
Reacting ω-trifluoroacetamidoalkyl 2-acetamido-6-O-benzyl-2-deoxy-4-O-[β-D-galactopyranosyl]-β-D-glucopyranoside (A) with 2,2-dimethoxypropane in acidic conditions to provide an intermediate reaction product;
Neutralisation of the intermediate reaction product followed by acetylation to provide a final reaction product; and then
Precipitation of the quantity from the final reaction product.

Preferably the ω-trifluoroacetamidoalkyl moiety is a ω-trifluoroacetamido-C₂₋₄-alkyl moiety. More preferably the ω-trifluoroacetamidoalkyl moiety is a 3-trifluoroacetamidopropyl moiety.

Preferably the reacting with 2,2-dimethoxypropane is with methanol as a catalyst. Preferably the acidic conditions are provided by camphor sulfonic acid. Preferably the neutralisation is by pyridine.

Preferably the quantity of the at least 90% (w/w) ω-trifluoroacetamidoalkyl 2-acetamido-6-O-benzyl-2-deoxy-4-O-[2,6-di-O-acetyl-3,4-O-isopropylidene-β-D-galactopyranosyl]-β-D-glucopyranoside (F) is greater than 10 g. More preferably the quantity is greater than 30 g.

In an embodiment of the fourth aspect a method of preparing at least 90% (w/w) 3-trifluoroacetamidopropyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (12) is provided, the method comprising:
Incubating in the presence of a catalytic amount of methanol a suspension of 3-trifluoroacetamidopropyl 2-acetamido-6-O-benzyl-2-deoxy-4-O-[β-D-galactopyranosyl]-β-D-glucopyranoside (11) in 2,2-dimethoxypropane acidified with camphorsulfonic acid at a temperature and for a time sufficient to provide a 3′4′-O-isopropylidenated intermediate reaction product;
Neutralising the intermediate reaction product with pyridine to provide a neutralised intermediate reaction product;
Concentrating the neutralised intermediate reaction product to provide a concentrated intermediate reaction product;
Incubating a mixture of acetic anhydride, the concentrated intermediate reaction product, and pyridine, at a temperature and for a time sufficient to provide a final reaction product comprising 3-trifluoroacetamidopropyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (12);
Concentrating the final reaction product to provide a concentrated final reaction product; and then
Precipitating the greater than 10 g of at least 90% (w/w) 3-trifluoroacetamidopropyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (12) by the addition of hexane to a solution in diethyl ether of the concentrated final reaction product.

Preferably the precipitate of the greater than 10 g of at least 90% (w/w) 3-trifluoroacetamidopropyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (12) is crystalline.

In a fifth aspect a batch quantity of at least 90% (w/w) ω-trifluoroacetamidoalkyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (F) where the quantity is greater than 30 g is provided. Preferably the at least 90% (w/w) ω-trifluoroacetamidoalkyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (F) is crystalline.

Preferably the ω-trifluoroacetamidoalkyl moiety is a ω-trifluoroacetamido-C₂₋₄-alkyl moiety. More preferably the ω-trifluoroacetamidoalkyl moiety is a 3-trifluoroacetamidopropyl moiety.

In a sixth aspect a method for the batch preparation of a quantity of a W-trifluoroacetamidoalkyl 2-acetamido-3-O-acetyl-6-O-benzyl-2-deoxy-4-O-[2,6-di-O-acetyl-β-D-galactopyranosyl]-β-D-glucopyranoside (G) of the following structure is provided:

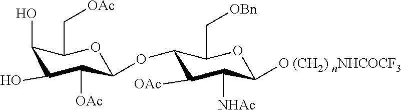

where n is the integer 2, 3, 4, 5, 6, 7 or 8. Preferably the quantity is greater than 10 g. More preferably the quantity is greater than 30 g.

The method comprises:
Dissolving a ω-trifluoroacetamidoalkyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (F) in a first volume of organic solvent and mixing with a second volume of water to provide an emulsion; and then
Adding a third volume of an acid to the emulsion and stirring at a temperature and for a time sufficient to provide a reaction product comprising the ω-trifluoroacetamidoalkyl 2-acetamido-3-O-acetyl-6-O-benzyl-2-deoxy-4-O-[2,6-di-O-acetyl-β-D-galactopyranosyl]-β-D-glucopyranoside (G).

Typically, the reaction product is diluted in a fourth volume of a saturated aqueous bicarbonate solution, the diluted reaction product extracted with volumes of organic solvent, and the combined volumes of organic solvent concentrated in vacuo to provide the quantity of the ω-trifluoroacetamidoalkyl 2-acetamido-3-O-acetyl-6-O-benzyl-2-deoxy-4-O-[2,6-di-O-acetyl-β-D-galactopyranosyl]-β-D-glucopyranoside (G).

Preferably the dissolving a ω-trifluoroacetamidoalkyl 2-acetamido-6-O-benzyl-2-deoxy-4-O-[2,6-di-O-acetyl-3,4-O-isopropylidene-β-D-galactopyranosyl]-β-D-glucopyranoside (F) is the dissolving of a crystalline form of the W-trifluoroacetamidoalkyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (F).

Preferably the ω-trifluoroacetamidoalkyl moiety is a ω-trifluoroacetamido-C₂₋₄-alkyl moiety. More preferably the ω-trifluoroacetamidoalkyl moiety is a 3-trifluoroacetamidopropyl moiety.

Preferably the first volume of organic solvent is a first volume of chloroform. More preferably the ratio of the first volume to the second volume is in the range 90:1 to 110:1. Most preferably the ratio of the first volume to the second volume is 100:1.

Preferably the third volume of an acid is a third volume of trifluoroacetic acid (TFA). More preferably the ratio of the first volume to the third volume is 9:1 to 11:1. Most preferably the ratio of the first volume to the third volume is 10:1.

In an embodiment of the sixth aspect a method of preparing 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13) is provided, the method comprising:
Dissolving 3-trifluoroacetamidopropyl 2-acetamido-3-O-acetyl-6-O-benzyl-2-deoxy-4-O-[2,6-di-O-acetyl-3,4-O-isopropylidene-β-D-galactopyranosyl]-β-D-glucopyranoside (12) in chloroform followed by the addition of about 1% (v/v) water with agitation to form an emulsion;
Adding about 10% (v/v) trifluoroacetic acid to the emulsion and stirring at a temperature and a time sufficient to provide a reaction mixture comprising 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13);
mixing the reaction mixture with a 2- to 4-fold excess volume of aqueous saturated bicarbonate solution to provide an organic layer and an aqueous volume;
Extracting the aqueous volume with at least one volume of ethyl acetate;
Combining the organic layer and the at least one volume of ethyl acetate to provide a combined organic volume;
Drying and then reducing the volume of the combined organic volume to provide a reduced volume; and then
Precipitating the quantity of 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13) by addition of a volume of hexane to the reduced volume.

The precipitated 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13) may be used in a method of preparing 3-trifluoroacetamidopropyl 2-acetamido-3-O-acetyl-6-O-benzyl-2-deoxy-4-O-[2,4,6 tri-O-acetyl-β-D-galactopyranosyl]-β-D-glucopyranoside (14) without further purification.

In a seventh aspect a method for the batch preparation of a quantity of ω̅-trifluoroacetamidoalkyl 2-acetamido-3-O-acetyl-6-O-benzyl-2-deoxy-4-O-[2,4,6 tri-O-acetyl-β-D-galactopyranosyl]-β-D-glucopyranoside (C) of the following structure is provided:

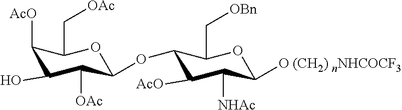

where n is the integer 2, 3, 4, 5, 6, 7 or 8. Preferably the quantity is greater than 10 g. More preferably the quantity is greater than 30 g.

The method comprises the crystallisation of the ω̅-trifluoroacetamidoalkyl 2-acetamido-3-O-acetyl-6-O-benzyl-2-deoxy-4-O-[2,4,6-tri-O-acetyl-β-D-galactopyranosyl]-β-D-glucopyranoside (C) from a solution in hexane of the residue obtained by the selective acetylation of the 4′-OH group of a ω̅-trifluoroacetamidoalkyl 2-acetamido-3-O-acetyl-6-O-benzyl-2-deoxy-4-O-[2,6-di-O-acetyl-β-D-galactopyranosyl]-β-D-glucopyranoside (G).

Preferably the selective acetylation of the 4′-OH group is by triethyl orthoacetate in the presence of p-toluene sulfonic acid.

In a first embodiment of the seventh aspect a method of preparing 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14) is provided, the method comprising:
Dissolving 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13) in dry acetonitrile and adding triethyl orthoacetate and p-toluene sulfonic acid to provide a reaction mixture;
Incubating the reaction mixture at a temperature and for a time sufficient to provide a first reaction product comprising 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14);
Neutralising the reaction product with pyridine and reducing the volume of the neutralised reaction product to provide a first residue;
Dissolving the first residue in 80% aqueous acetic acid and incubating at a temperature and for a time sufficient to provide a second reaction product comprising 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14);
Reducing the volume of the second reaction product to provide a second residue; and then
Precipitating the quantity of 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14) by the addition of a volume of hexane to a solution in ethyl acetate of the second residue.

In a second embodiment of the seventh aspect a method for the batch preparation of 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14) as a crystalline product is provided. The method comprises:
Deacetylation of 3-trifluoroacetamidopropyl 2-acetamido-3-O-acetyl-6-O-benzyl-2-deoxy-4-O-[2,3,4,6-tetra-O-acetyl-α/β-D-galactopyranosyl]-β-D-glucopyranoside (10) to provide a mixture of 3-trifluoroacetamidopropyl 2-acetamido-6-O-benzyl-2-deoxy-4-O-[β-D-galactopyranosyl]-β-D-glucopyranoside (11) and its anomer 3-fluoroacetamidopropyl 2-acetamido-6-O-benzyl-2-deoxy-4-O-[α-D-galactopyranosyl]-β-D-glucopyranoside;
Crystallisation of 3-trifluoroacetamidopropyl 2-acetamido-6-O-benzyl-2-deoxy-4-O-[β-D-galactopyranosyl]-β-D-glucopyranoside (11) from a solution of the mixture to provide 3-trifluoroacetamidopropyl 2-acetamido-6-O-benzyl-2-deoxy-4-O-[β-D-galactopyranosyl]-β-D-glucopyranoside (11) as a first crystalline intermediate;
Isopropylidenation of the first crystalline intermediate using 2,2-dimethoxypropane in acidic conditions followed by acetylation using acetic anhydride and pyridine to provide a residue comprising 3-trifluoroacetamidopropyl 2-acetamido-6-O-benzyl-2-deoxy-4-O-[2,6-di-O-acetyl-3,4-O-isopropylidene-β-D-galactopyranosyl]-β-D-glucopyranoside (12);
Crystallisation of 3-trifluoroacetamidopropyl 2-acetamido-6-O-benzyl-2-deoxy-4-O-[2,6-di-O-acetyl-3,4-O-isopropylidene-β-D-galactopyranosyl]-β-D-glucopyranoside (12) from a solution of the residue to provide 3-trifluoroacetamidopropyl 2-acetamido-6-O-benzyl-2-deoxy-4-O-[2,6-di-O-acetyl-3,4-O-isopropylidene-β-D-galactopyranosyl]-β-D-glucopyranoside (12) as a second crystalline intermediate;
Deisopropylidenation of the second crystalline intermediate using trifluoroacetic acid and precipitation of the product of the reaction to provide a precipitate comprising 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13);
Selective acetylation of 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (13) using triethyl orthoacetate to provide a residue comprising 3-trifluoroacetamidopropyl 2-acetamido-3-O-acetyl-6-O-benzyl-2-deoxy-4-O-[2,4,6-tri-O-acetyl-β-D-galactopryanosyl]-β-D-glucopyranoside (14); and then
Crystallisation of 3-trifluoroacetamidopropyl 2-acetamido-3-O-acetyl-6-O-benzyl-2-deoxy-4-O-[2,4,6-tri-O-acetyl-β-D-galactopryanosyl]-β-D-glucopyranoside (14) from a solution of the residue to provide the crystalline product.

The method corresponds to that presented in SCHEME II (commencing with 3-trifluoroacetamidopropyl 2-acetamido-3-O-acetyl-6-O-benzyl-2-deoxy-4-O-[2,3,4,6-tetra-O-acetyl-α/β-D-galactopyranosyl]-β-D-glucopyranoside (10)).

The 3-trifluoroacetamidopropyl 2-acetamido-3-O-acetyl-6-O-benzyl-2-deoxy-4-O-[2,4,6-tri-O-acetyl-β-D-galactopyranosyl]-β-D-glucopyranoside (14) may be used in the preparation of 3-trifluoroacetamidopropyl glycosides (D) of the following structure:

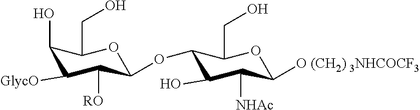

where R is H or Glyc and Glyc is a glycan. Preferably Glyc is a monosaccharide residue. More preferably R is H and Glyc is the monosaccharide residue α-D-galactopyranosyl.

The 3-trifluoroacetamidopropyl glycosides (D) can be quantitatively converted to the corresponding ω-aminopropyl glycosides (E) by an aqueous base. Examples of ω-aminopropyl glycosides are Galα3Galβ4GlcNAcβ-O(CH₂)₃NH₂ (Bₜᵣᵢ(type 2); Galili), GalNAcα3(Fucα2)Galβ4GlcNAcβ-O(CH₂)₃NH₂ (A (type 2)), Galα3(Fucα2)Galβ4GlcNAcβ-O(CH₂)₃NH₂ (B (type 2)), Fucα2Galβ4GlcNAcβ-O(CH₂)₃NH₂ (H (type 2)), Galβ4(Fucα3)GlcNAcβ-O(CH₂)₃NH₂ (Le^{x}), Fucα2Galβ4(Fucα3)GlcNAcβ-O(CH₂)₃NH₂ (Le^{y}), and Galα4Galβ4GlcNAcβ-O(CH₂)₃NH₂ (Pᵢ).

In a ninth aspect a method for the batch preparation of an ω-aminoalkyl tri-, tetra- or oligosaccharide (ω-aminoalkyl glycoside) of the following structure is provided:

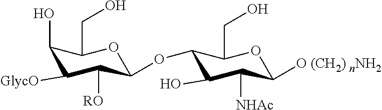

where R is H or Glyc, Glyc is a glycan, and n is the integer 2, 3, 4, 5, 6, 7 or 8.

Preferably the ω-aminoalkyl glycoside is a 2-aminoethyl glycoside, a 3-aminopropyl glycoside, or a 4-aminobutyl glycoside. More preferably the W-aminoalkyl glycoside is a 3-aminopropyl glycoside.

The method comprises precipitating from a solution in an organic solvent a quantity of a corresponding ω-trifluoroacetamidoalkyl 2-acetamido-3-O-acetyl-6-O-benzyl-2-deoxy-4-O-[2,4,6-tri-O-acetyl-β-D-galactopryanosyl]-β-D-glucopyranoside (C). Preferably the quantity is greater than 10 g. More preferably the quantity is greater than 30 g.

Preferably the precipitating is the crystallising from the solution in the organic solvent the quantity of the ω-trifluoroacetamidoalkyl 2-acetamido-3-O-acetyl-6-O-benzyl-2-deoxy-4-O-[2,4,6-tri-O-acetyl-β-D-galactopryanosyl]-j-D-glucopyranoside (C).

Preferably Glyc is a monosaccharide residue. More preferably, Glyc is a monosaccharide residue independently selected from the group consisting of: α-D-galactopyranosyl (Gal); 2-acetamido-α-D-galactopyranosyl (GalNAc); and L-fucopyranosyl (Fuc). Most preferably, R is H and Glyc is the monosaccharide residue α-D-galactopyranosyl.

In a tenth aspect a method for the batch preparation of a quantity of the glycoconjugate AGI-134 (CAS RN 1821461-84-0) (22) is provided, the method comprising the use of any one or more of the preceding aspects. Preferably the quantity is greater than 1 g. More preferably the quantity is greater than 3 g. Most preferably the quantity is greater than 10 g. Preferably, the method excludes the use of column chromatography other than as an ultimate step in the purification of the glycoconjugate.

The methods obviate the need to purify the intermediates or products by column chromatography. The methods circumvent the need for column chromatography and consequential reductions in yield by proceeding via intermediates and products that may be isolated in substantially pure or useable purity by crystallisation or precipitation. The methods also avoid the use of toxic reagents (such as cyanide and salts of mercury) and inert atmospheres. These advantages make the methods eminently suitable for use at a preparative scale.

In the description and claims of this specification the following abbreviations, acronyms, terms and phrases have the meaning provided: “Ac” denotes acetyl; “anomer” means an epimer at the hemiacetal/hemiketal carbon in a cyclic saccharide; “anomeric mixture” means a mixture of anomers; “Aₜₑₜᵣₐ” denotes GalNAcα3(Fucα2)Galβ4GlcNAc; “Aₜᵣᵢ” denotes GalNAcα3(Fucα2)Gal(3; “batch preparation” means the preparation of a quantity at one time, i.e., in a single production run; “batch quantity” means a quantity prepared at one time, i.e., in a single production run; “Bn” denotes benzyl; “Bₜₑₜᵣₐ” denotes Galα3(Fucα2)Galβ4GlcNAc; “Bₜᵣᵢ” denotes Galα3(Fucα2)Galβ; “CAS RN” means Chemical Abstracts Service (CAS, Columbus, Ohio) Registry Number; “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “Fuc” denotes fucose (L-fucopyranose or L-fucopyranosyl); “Gal” denotes galactose (D-galactopyranose or D-galactopyranosyl); “Galili” denotes Galα3Galβ4GlcNAc; “GalN” denotes galactosamine (2-amino-2-deoxy-D-galactopyranose or 2-amino-2-deoxy-D-galactopyranosyl); “GalNAc” denotes N-acetylgalactosamine (2-acetamido-2-deoxy-D-galactopyranose or 2-acetamido-2-deoxy-D-galactopyranosyl); “Glc” denotes glucose (D-glucopyranose or D-glucopyranosyl); “GlcN” denotes glucosamine (2-amino-2-deoxy-D-glucopyranose or 2-amino-2-deoxy-D-glucopyranosyl); “GlcNAc” denotes N-acetylglucosamine (2-acetamido-2-deoxy-D-glucopyranose or 2-acetamido-2-deoxy-D-glucopyranosyl); “Gly” denotes a monosaccharide; “Glyc” denotes a glycan comprising one or more monosaccharide units, i.e., a monosaccharide, disaccharide, trisaccharide or oligosaccharide; “LacN” denotes lactosamine (Galβ4GlcN); “LacNAc” denotes N-acetyllactosamine (Galβ4GlcNAc); “oligosaccharide” means four or more saccharide residues; “Ph” denotes phenyl; “preparative scale” means prepared as a batch of greater than 100 mg; and “sp” denotes ω-aminoalkyl, i.e., —(CH₂)ₙNH₂ where n is an integer from 2 to 8 inclusive. A paronym of any of the defined terms has a corresponding meaning. “Benzylidene” and “phenylmethylene” are equivalent terms.

The terms “first”, “second”, “third”, etc. used with reference to elements, features, integers, or other limitations, of the matter described in the Summary of Invention, or when used with reference to alternative aspects or embodiments are not intended to imply any order of preference. Elements, features, integers, or other limitations, of the elements described in the Summary of Invention are identified in order of preference by the introductory “preferably . . . ”, “more preferably . . . ”, “yet more preferably . . . ” and so on. Preferred combinations of elements, features, integers, or other limitations, of the matter described in the Summary of Invention are similarly identified.

Where concentrations or ratios are specified the concentration or ratio specified is the initial concentration or ratio. Similarly, where a pH or pH range is specified, the pH or pH range specified is the initial pH or pH range. Where values are expressed to one or more decimal places standard rounding applies. For example, 1.7 encompasses the range 1.650 recurring to 1.749 recurring.

Where a parameter is expressed as being “about” a specified range or value the term is used to indicate tolerance for some variation of the specified range or value (with the proviso that the parameter dependent effect is still achieved). In the absence of any other proviso the term “about” should be understood to indicate a tolerance of no greater than 5% above or below the upper and lower limits, respectively, of the specified range or plus or minus 5% of the specified value.

A representation of the structure of the disaccharide derivative W-trifluoroacetamidoalkyl 2-acetamido-3-O-acetyl-6-O-benzyl-2-deoxy-4-O-[2,4,6-tri-O-acetyl-β-D-galactopyranosyl]-β-D-glucopyranoside (B) (where n is an integer in the range 1 to 8) is:

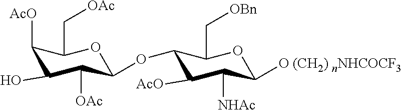

This disaccharide derivative may alternatively be named ω-trifluoroacetamidoalkyl O-(2,4,6-tri-O-acetyl-β-D-galactopyranosyl)-(1-4)-2-acetamido-3-O-acetyl-6-O-benzyl-2-deoxy-β-D-glucopyranoside. Similar alternative naming conventions may be adopted for the other di-, tri- and oligosaccharide derivatives described here.

Where required, the following convention for identifying the carbons of N-acetyllactosamine (Galβ4GlcNAc) is also adopted:

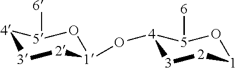

For example, the designation 3′-OH identifies the hydroxyl group located at carbon 3′ of the galactopyranosyl substituent of N-acetyllactosamine. Similarly, the galactopyranosyl substituent is located at carbon 4 of the 2-acetamido-2-deoxy-glucopyranoside of N-acetyllactosamine.

The invention will now be described with reference to embodiments or examples.

### DESCRIPTION

Known synthetic methods of preparing N-acetyllactosamine (Galβ4GlcNAc) derivatives either: (i) start with a precursor comprising a disaccharide with the required (4 glycosidic bond, or (ii) form this bond by reacting a suitable glycosyl donor with a suitable glycosyl acceptor. In the latter case, a glycosyl donor is reacted with a glycosyl acceptor which has only one free hydroxyl group. The protecting (a.k.a. “blocking”) system for both of the reactants must be assessed. The reactivity of both the glycosyl donor and the glycosyl acceptor depend to a high degree on the protecting system and the further substituents of the two reactants. As stated in the publication of Paulsen (1985), it is not possible to indicate standard conditions for an oligosaccharide synthesis, but each glycosidic bond forming reaction must be optimized according to the problem being addressed. In the present case, the problem being addressed is the provision of a readily scalable method of preparing N-acetyllactosamine derivatives suitable for use in the preparation of glycoconjugates. In general terms, for a multi-step synthetic method, this means obviating the requirement for column chromatography wherever possible.

Known methods of preparing saccharides comprising a terminal D-galactopyranosyl group with a single unprotected 3-OH group, such as those disclosed in the publications of Severov et al (2007) and Rhyzov et al (2019), often employ the use of a borohydride (e.g., sodium cyanoborohydride (NaBH₃CN)) in the reduction of the corresponding benzylidene intermediate. The method described here avoids the use of borohydrides and therefore the formation of boron esters that might otherwise frustrate purification of the intermediates by crystallisation or precipitation.

The realisation of the advantage of scalability by obviating the need for column chromatography is attributed at least in part to the formation of the fully protected disaccharide intermediate 3-trifluoroacetamidopropyl 2-acetamido-3-O-acetyl-6-O-benzyl-2-deoxy-4-O-[2,3,4,6-tetra-O-acetyl-α/β-D-galactopyranosyl]-β-D-glucopyranoside (α/β-Ac4Gal-β-6-O-Bn-AcGlcNAc-sp3). This disaccharide intermediate has a unique combination of protecting groups, including a single 6-O-benzyl group. The glycosidic bond of the anomeric mixture is formed by reacting the glycosyl donor 1-thioethyl-2,3,4,6-tetra-O-acetyl-β-D-galactopyranoside (β-Ac₄GalSEt) with the glycosyl acceptor 3-trifluoroacetamidopropyl 2-acetamido-3-O-acetyl-6-O-benzyl-2-deoxy-β-D-glucopyranoside (β-6-O-Bn-3-AcGlcNAc-sp3). Both of these reactants are also preparable by chromatography-free methods as described here.

Chromatography-free methods of preparing the fully protected disaccharide derivative 3-trifluoroacetamidopropyl 2-acetamido-6-O-benzyl-2-deoxy-4-O-[2,6-di-O-acetyl-3,4-O-isopropylidene-β-D-galactopyranosyl]-β-D-glucopyranoside (β-2′,6′-O-Ac-3′,4′-O-iPdGal-β-6-O-Bn-3-O-AcGlcNAc-sp3) and the partially protected disaccharide derivatives 3-trifluoroacetamidopropyl 2-acetamido-6-O-benzyl-2-deoxy-4-O-[β-D-galactopyranosyl]-β-D-glucopyranoside (β-Gal-β-6-O-Bn-GlcNAc-sp3), 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (β-2′,6′-O-AcGal-β-6-O-Bn-3-O-AcGlcNAc-sp3) and 3-trifluoroacetamidopropyl 2-acetamido-3-O-acetyl-6-O-benzyl-2-deoxy-4-O-[2,4,6-tri-O-acetyl-β-D-galactopryanosyl]-β-D-glucopyranoside (β-2′,4′,6′-O-AcGal-β-6-O-Bn-3-O-AcGlcNAc-sp3) are also provided. Each of these disaccharide derivatives is a 6-O-benzyl-glucopyranoside. Whilst not wishing to be bound by theory, the facility with which the derivatives may be crystallized may be attributed to this common structural feature.

As the methods of preparing partially protected N-acetyllactosamine derivatives described here provide the basis for scalable methods of preparing 3′-O-glycosyl and 2′,3′-O-di-glycosyl N-acetyllactosamine glycoconjugates, e.g., the clinical candidate AGI-134. Commencing with the partially protected disaccharide derivative 3-trifluoroacetamidopropyl 2-acetamido-3-O-acetyl-6-O-benzyl-2-deoxy-4-O-[2,4,6-tri-O-acetyl-β-D-galactopryanosyl]-β-D-glucopyranoside, the preparation of the glycan moieties of AGI-134 and other significant glycoconjugates is described in the publication of Kunetskiy et al (2020). In addition to Galα3Galβ4GlcNAcβ-OR (Bₜᵣᵢ(type 2); Galili), the partially protected N-acetyllactosamine derivatives described here may be used in the preparation of the following glycan moieties: GalNAcα3(Fucα2)Galβ4GlcNAcβ-OR (A (type 2)); Galα3(Fucα2)Galβ4GlcNAcβ-OR (B (type 2)); Fucα2Galβ4GlcNAcβ-OR (H (type 2)); Galβ4(Fucα3)GlcNAcβ-OR (Le^{x}); Fucα2Galβ4(Fucα3)GlcNAcβ-OR (Le^{y}); and Galα4Galβ4GlcNAcβ-OR (Pᵢ), where R is an aminoalkyl such as aminopropyl ((CH₂)₃NH₂)

### Experimental

Unless otherwise noted (see below), reagents and solvents were obtained from commercial suppliers and used without purification. Acetic acid and acetic anhydride (Ac₂O) were refluxed and distilled over dry sodium acetate (NaOAc) Chloroorganic solvents were refluxed and distilled over phosphorus pentoxide (P₂O₅). If absolute chloroorganic solvent was needed, the solvent was additionally distilled over calcium hydride (CaH₂) in dry atmosphere. Pyridine was refluxed and distilled over sodium hydroxide (NaOH). Absolute methanol was refluxed and distilled over magnesium (Mg). Reactants such as trifluoroacetic-3-aminopropanol (TFA-3-aminopropanol), 1-thioethyl-2,3,4,6-tetra-O-acetyl-β-D-galactopyranoside (β-Ac₄GalSEt) and 1-thioethyl-2,3,4,6-tetra-O-benzyl-β-D-galactopyranoside (β-Bn₄GalSEt) were prepared according to chromatography free methods.

TLC was performed on pre-coated aluminium plates (Silica Gel F₂₅₄). Spots were visualized by drying with mild heating, immersion in 10% aqueous phosphoric acid (H₃PO₄) followed by heating at 200° C. or more. All NMR spectra were recorded on a 400 MHz spectrometer. Proton chemical shifts are reported in parts per million (ppm) using deuterated chloroform (CDCl₃), deuterated methanol (CD₃OD) or deuterated dimethylsulfoxide ((CD₃)₂SO) residual peaks as internal references.

### Preparation of 3-trifluoroacetamidopropyl 2-acetamido-3,4,6-tri-O-acetyl-2-deoxy-β-D-glucopyranoside (β-Ac3GlcNAc-sp3) (4)

Intermediate β-Ac3GlcNAc-sp3 (4) was prepared according to SCHEME IA. An aminoalkyl triacetylated monosaccharide where the substituted hydroxyl groups can be selectively deprotected is provided.

Glucosamine hydrochloride (GlcNH₃Cl) (1) (160 g, 0.75 mol) was suspended in a mixture of pyridine (500 mL) and Ac₂O (500 mL, 5.35 mol). The reaction mixture was stirred at 60° C. for two days. The mixture was cooled down to room temperature (r.t.), and any residual Ac₂O slowly quenched with water (100 mL).

The solution was concentrated on rotary evaporator, co-evaporated with water (500 mL) and dried in vacuo. The residual dark oil was dissolved in chloroform (1.2 L), washed with 1N HCl (1 L), and then washed with water (0.5 L). The aqueous layers were combined and extracted with chloroform (0.5 L). The combined organic layers were washed with cold aqueous saturated sodium bicarbonate (NaHCO₃) (1 L), brine (0.5 L), and dried over anhydrous sodium sulfate (Na₂SO₄) (100 mL powder)/silicon dioxoide (SiO₂) (50 mL powder). The resulting solution of 2-acetamido-3,4,6-tetra-O-acetyl-2-deoxy-D-glucopyranosyl acetate (Ac₄GlcNAc) (2) was used without additional treatment.

The solution of Ac₄GlcNAc (2) in chloroform was cooled to 0° C. and acetyl bromide (AcBr) (175 mL, 2.4 mol) added. Water (40 mL, 2.2 mol) was added dropwise with stirring continued for 15 min. The reaction was left for 4 days at 0° C. The conversion was monitored by TLC (2:1 (v/v) chloroform/acetone). Pyridine (400 mL, 5 mol) was added dropwise at 0° C. and the reaction mixture then allowed to warm up to r.t. The conversion to oxazoline was monitored by

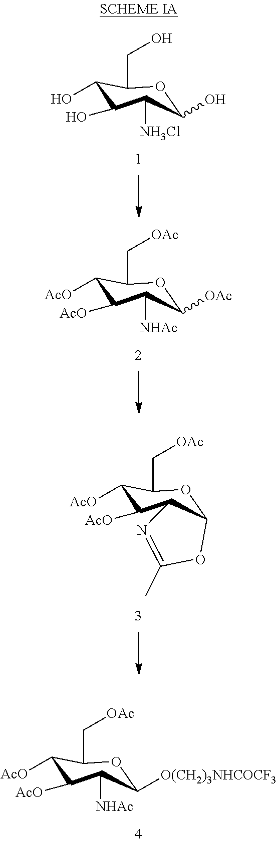

TLC (2:1 (v/v) chloroform/acetone). The mixture was poured in water (1 L), separated, and additionally washed with water (0.5 L). The combined aqueous layers were extracted with chloroform (0.5 L). The combined organic layers were washed with water (1 L), brine (0.5 L) and the dried over anhydrous Na₂SO₄ (100 mL powder). The solution was concentrated on a rotary evaporator, co-evaporated with toluene (3×0.5 L) and dried in vacuo. The crude Ac3GlcNAc-oxazoline (3) was used without additional purification. If required, the product could be stored at −15° C. for a few days.

A solution of 3-aminopropanol (0.45 L, 6 mol) in methanol (0.6 L) was cooled to 5° C. Methyl trifluoroacetate (CF₃COOMe) (0.7 L, 7 mol) was added dropwise. The reaction was left for 1 h and the pH of the reaction mixture monitored (neutral, otherwise additional amount of CF₃COOMe should be added). The reaction was left overnight, concentrated on a rotary evaporator, and the syrup obtained distilled in vacuo leading to the target TFA-3-aminopropanol (900 g): bp 90-100° C. (1 mm Hg). Yield: 90%. NMR spectra were in accordance with those disclosed in the publication of Bobkov et al (2008).

The crude Ac3GlcNAc-oxazoline (3) was dissolved in absolute dichloroethane (CH₂ClCH₂Cl) (1.2 L) followed by addition of TFA-3-aminopropanol (128 g, 0.75 mol), freshly dried MS-4 Å (80 g) and methanesulfonic acid (MsOH) (14 mL, 0.22 mol). The reaction mixture was stirred at 70° C. for 2 h and then at 60° C. overnight. The mixture was allowed to cool down to r.t. and then filtered through Celite. The solution obtained was washed with water (1 L) and the aqueous layer extracted with chloroform (0.5 L). the combined organic layers were washed with cold aq. saturated NaHCO₃ (1 L), brine (1 L) and then dried over anhydrous Na₂SO₄ (200 mL powder)/SiO₂ (100 mL powder). The solution was concentrated on rotary evaporator, co-evaporated with ethyl acetate and dried in vacuo at 40° C. The residue was dissolved in warm ethyl acetate (400 mL) and the product precipitated with hexane (400 mL) added drop-wise under intensive mechanical stirring. The mixture was left overnight for complete precipitation. The suspension was diluted with a mixture of 1:1 (v/v) hexane/ethyl acetate (400 mL) and the product collected on a filter. The product was washed with a mixture of 2:1 (v/v) hexane/ethyl acetate (1 L), 3:1 (v/v) hexane/ethyl acetate (1 L) and finally hexane (200 mL). The crystalline product was dried under a fume-hood overnight, ground to a fine powder with a spatula and dried in vacuo for a few hours at 40° C. TLC control (2:1 (v/v) chloroform/acetone). Crème-white powder of β-Ac3GlcNAc-sp3 (4) (168 g, 0.34 mol). Total yield for three steps: 45%. NMR spectra were in accordance with those disclosed in the publication of Bovin et al (1987).

### Preparation of 3-trifluoroacetamidopropyl 2-acetamido-3-O-acetyl-6-O-benzyl-2-deoxy-β-D-glucopyranoside (β-6-O-Bn-3-AcGlcNAc-sp3) (8)

Intermediate β-6-O-Bn-3-AcGlcNAc-sp3 (8) was prepared according to SCHEME IB. A glycosyl acceptor (monosaccharide) with a single deprotected 4-OH group and a benzylated 6-OH group is provided.

3-trifluoroacetamidopropyl 2-acetamido-3,4,6-tri-O-acetyl-2-deoxy-β-D-glucopyranoside (β-Ac₃GlcNAc-sp3) (4) (168 g, 0.34 mol) was dissolved in abs. methanol (2 L). 2M sodium methoxide (NaOMe) (20 mL, 0.04 mol) was then added, and the reaction left for 3 h. The conversion was monitored by TLC (3:1 (v/v) chloroform/methanol, Rf˜0.2, or 2:1 (v/v) chloroform/acetone, Rf˜0. The mixture was neutralised by Dowex50WX4 (20 g, H⁺-form) and filtered after 15 min of stirring. The solution was concentrated on a rotary evaporator, co-evaporated with acetonitrile (50 mL) and dried in vacuo. The crude 3-trifluoroacetamidopropyl 2-acetamido-2-deoxy-β-D glucopyranoside (β-GlcNAc-sp3) (5) was used without additional purification.

The crude β-GlcNAc-sp3 (5) was suspended in dry acetonitrile (1.5 L). Dimethoxymethylbenzene (PhCH(OMe)₂) (100 mL, 0.66 mol) and p-toluenesulfonic acid monohydrate (TsOH·H₂O) (6.4 g, 33 mmol) were then added. The reaction mixture was stirred overnight with a mechanical stirrer at r.t. and left for an additional 6 h without stirring at 0° C. The conversion was monitored by

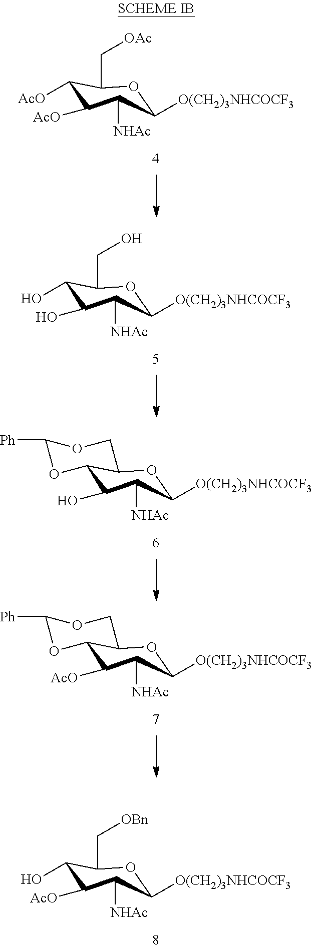

TLC (10:1 (v/v) chloroform/methanol, Rf˜0.3). The reaction was neutralised with pyridine (20 mL). The crystalline 3-trifluoroacetamidylpropyl 2-acetamido-4,6-O-phenylmethylene-2-deoxy-β-D-glucopyranoside (β-4,6-O-BdGlcNAc-sp3) (6) was collected on a glass filter, washed with a small portion of cold acetonitrile, dried in vacuo and used without additional purification.

The crystalline β-4,6-O-BdGlcNAc-sp3 (6) was mixed with pyridine (600 mL) and Ac₂O (300 mL). The mixture was stirred with a mechanical stirrer for two days. The conversion was monitored by TLC (10:1 (v/v) chloroform/methanol, Rf˜0.45). The suspension was diluted with 1:1 (v/v) hexane/ethyl acetate (900 mL). The crystalline product was collected on a glass filter, washed with 1:1 (v/v) hexane/ethyl acetate (2×500 mL), hexane (500 mL), and then dried under a fume-hood overnight by vacuum drying. White powder of 3-trifluoroacetamidopropyl 2-acetamido-3-O-acetyl 4,6-O-phenylmethylene-2-deoxy-β-D-glucopyranoside (β-4,6-O-Bd-3-O-AcGlcNAc-sp3) (7) (136 g, 0.27 mol). Total yield for last three steps: 80%. ¹H NMR ((CD₃)SO): δ 1.68-1.75 (m, 2H, CCH₂C), 1.78, 1.96 (2s, 6H, OC(O)CH₃, NHC(O)CH₃), 3.14-3.25 (m, 2H, CH₂N), 3.45-3.54 (m, 2H, CH₂O), 3.70-3.82 (m, 4H, H-2, H-4, H-5, H-6a), 4.24 (dd, 1H, J_{6b,5} 5.0, J_{6b,6a} 10.2, H-6b), 4.65 (d, 1H, J_{1,2} 8.4, H-1), 5.12 (dd, 1H, J_{3,2}˜J_{3,4}˜10.0, H-3), 5.63 (s, 1H, CHPh), 7.35-7.40 (m, 5H, ArH), 7.91 (d, 1H, J_{NH,2} 9.3, NHAc), 9.34 (br t, 1H, NHC(O)CF₃).

To a 2 L flask containing freshly dried MS-4 Å (50 mL, granules) abs. dichloromethane (600 mL) was added. After 15 min of stirring, the reaction flask was cooled down to 0° C. A solution of β-4,6-O-Bd-3-O-AcGlcNAc-sp3 (7) (136 g, 0.27 mol), triethylsilane (Et₃SiH) (260 mL, 1.62 mol) and trifluoroacetic acid (TFA) (103 mL, 1.35 mol) in dry dichloromethane (120 mL) was added. After dissolving of the starting material, the reaction flask was closed with a calcium chloride (CaCl₂) tube, left for 12 h at 0° C. and additionally for 12 h at r.t. The conversion was monitored by TLC (10:1 (v/v) chloroform/methanol, Rf˜0.3). The reaction mixture was slowly poured into a 5 L beaker with aq. saturated sodium bicarbonate (NaHCO₃) (2 L) under intensive mechanical stirring. The organic layer was separated, the aqueous layer extracted with chloroform (250 mL), and the combined organic layers washed with water (500 mL). The aqueous layer was extracted with chloroform (3×250 mL). All the organic layers were combined and dried over anhydrous sodium sulfate (Na₂SO₄) (200 mL powder). The solution was concentrated on a rotary evaporator, co-evaporated with ethyl acetate, and dried to viscous oil in vacuo. The residue was dissolved in ethyl acetate (600 mL) and for precipitation of the product hexane (1.2 L) was added dropwise under mechanical stirring. The stirring was turned off and the suspension left overnight for complete precipitation. The crystalline product was collected on a glass filter, washed with a mixture of 4:1 (v/v) hexane/ethyl acetate (2×500 mL). For additional purification the obtained product was dissolved in ethyl acetate (600 mL) under warming, cooled down to r.t., and precipitation repeated. The crystalline product was collected on a glass filter, washed with a mixture of 4:1 (v/v) hexane/ethyl acetate (500 mL) followed by pure hexane (500 mL), and dried under a fume-hood overnight by vacuum drying. White powder of β-6-O-Bn-3-AcGlcNAc-sp3 (8) (80 g, 0.158 mol). Yield: 60%. NMR spectra were in accordance with those disclosed in the publications of Bovin et al (1988) and Zinin et al (2007). ¹H NMR ((CD₃)₂SO): δ 1.73 (m, 2H, CCH₂C), 1.74 (s, 3H, NH(O)CH₃), 1.95 (s, 3H, C(O)CH₃), 3.14-3.25 (m, 2H, NCH₂), 3.32 (ddd, 1H, J_{2,1}˜J_{2,3}˜J_{2,NH}˜8.9, H-2), 3.39-3.50 (m, 2H, CH₂O), 3.55-3.81 (2m, 4H, H-4, H-5, 2H-6), 4.46 (d, 1H, J_{1,2} 8.5, H-1), 4.53 (s, 2H, CH₂Ph), 4.82 (t, 1H, J_{3,4}˜J_{3,2}˜9.8, H-3), 5.40 (d, 1H, J_{OH,4} 6.2, OH), 7.22-7.40 (m, 5H, ArH), 7.78 (d, 1H, J_{NH,2} 9.2, NHAc), 9.33 (br t, 1H, NHC(O)CF₃).

### Preparation of 3-trifluoroacetamidopropyl 2-acetamido-3-O-acetyl-6-O-benzyl-2-deoxy-4-O-[2,4,6-tri-O-acetyl-β-D-galactopryanosyl]-β-D-glucopyranoside (β-2′,4′,6′-O-AcGal-β-6-O-Bn-3-O-AcGlcNAc-sp3) (14)

Target β-2′,4′,6′-O-AcGal-β-6-O-Bn-3-O-AcGlcNAc-sp3 (14) was prepared according to SCHEME II. A glysoyl acceptor (disaccharide) with a single deprotected 3′-OH group is provided.

To solution of β-galactose pentaacetate (115 g, 0.3 mol) in abs. dichloromethane (200 mL) freshly dried MS-4 Å (50 mL) was added and the reaction mixture cooled to 0° C. Ethanethiol (EtSH) (45 mL, 0.6 mol) was added, and the mixture stirred 15 min followed by addition of boron trifluoride diethyl etherate (Et₂O·BF₃) (42 mL, 0.35 mol) at 0° C. The reaction mixture was allowed to warm to r.t. and left for 24 h. The conversion was monitored by TLC (3:2 (v/v) toluene/ethyl acetate, Rf˜0.58). The reaction mixture was neutralized with pyridine (15 mL) and filtered over Celite. The solution was poured into aq. saturated sodium bicarbonate (NaHCO₃) (1.5 L), washed with an additional portion of aq. saturated sodium bicarbonate (NaHCO₃) (0.5 L), water (0.5 L), 1N HCl (1 L), water (0.5 L), once again with aq. saturated sodium bicarbonate (NaHCO₃) (0.5 L) and dried with sodium sulfate (Na₂SO₄). The solution obtained was concentrated on a rotary evaporator and dried in vacuo. The residual oil was diluted with diethyl ether (100 mL) and the solution was added dropwise to hexane (500 mL) under intensive stirring. The crystalline product was collected on a glass filter, washed with a cold mixture of 5:1 (v/v) hexane/diethyl ether (200 mL), hexane (200 ml) and dried in vacuo. 1-thioethyl-2,3,4,6-tetra-O-acetyl-β-D-galactopyranoside (β-Ac₄GalSEt) (9) (95 g, 0.24 mol). Yield: 80%. NMR spectra were in accordance with those disclosed in the publication of Weïwer et al (2008). ¹H NMR (CDCl₃) δ 1.29 (t, 3H, J 7.5, SCH₂CH₃), 1.99, 2.06, 2.09, 2.18 (4s, 12H, 4C(O)CH₃), 2.79-2.68 (m, 2H, SCH₂CH₃), 3.94 (ddd, 1H, J_{5,6a}˜J_{5,6b} 6.7, J_{5,4} 1.2, H-5), 4.12 (dd, 1H, J_{6a,6b} 11.3, J_{6a,5} 6.6, H-6a) 4.19 (dd, 1H, J_{6b,6a} 11.3, J_{6b,5} 6.7, H-6b), 4.50 (d, 1H, J_{1,2} 10.0, H-1), 5.06 (dd, 1H, J_{3,4} 10.0, J_{3,2} 3.4, H-3), 5.25 (t, 1H, J 10.0, H-2), 5.44 (dd, 1H, J_{4,5} 3.4, J_{4,3} 1.2, H-4).

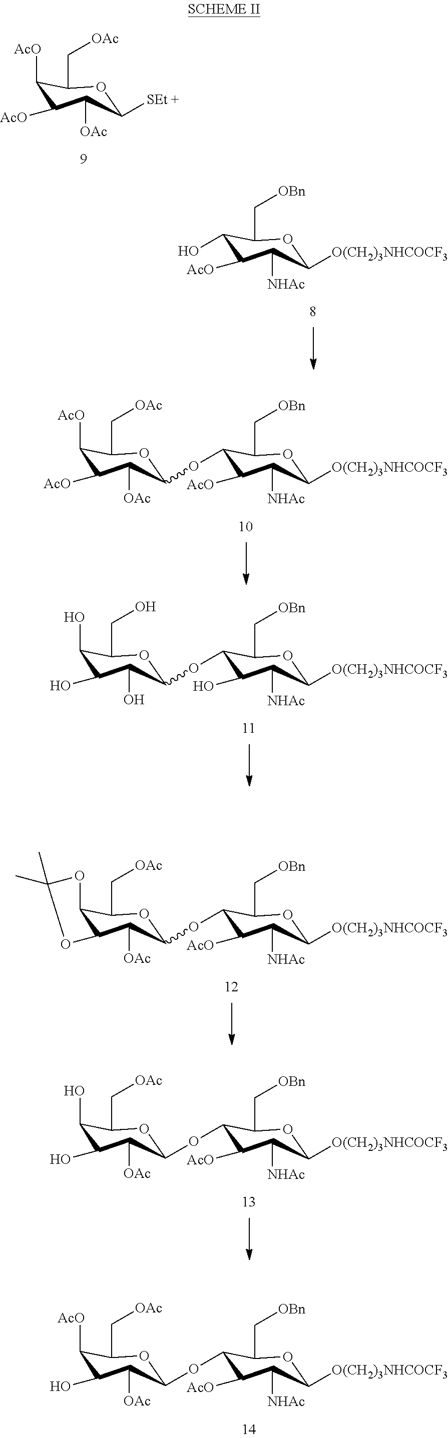

To a 4 L flask equipped with a dropping funnel β-3-O-Ac-6-O-BnGlcNAc-sp3 (8) (80 g, 0.158 mol), β-Ac₄GalSEt (9) (99 g, 0.253 mol) and freshly dried MS-4 Å (300 mL, granules) were added. The flask was filled with argon and abs. dichloromethane (3 L) added. The reaction mixture was stirred for 30 min.

The reaction mixture was cooled down to −10° C., NIS (60 g, 2 mol) added, and the mixture stirred for an additional 30 min. The reaction mixture was then cooled to −45° C. (acetonitrile/liquid nitrogen bath) and trifluoromethane sulfonic acid (TfOH) (28 mL, 0.316 mol) added dropwise. The reaction mixture was stirred at −45° C. for 2 h, then slowly warmed to −30° C. The conversion was monitored by TLC (10:1 (v/v) chloroform/methanol Rf(α)˜0.49, Rf(β)˜0.44).

The reaction mixture was neutralized with pyridine (80 mL) and filtered over Celite. The solution obtained was poured into 1M sodium thiosulfate (Na₂S₂O₃) (1 L), washed with cold aq. saturated sodium bicarbonate (NaHCO₃) (1 L) and dried over sodium sulfate/silicon dioxide (Na₂SO₄/SiO₂). The solution was concentrated on a rotary evaporator, hexane (500 mL) was added and the mixture was stirred at 40° C. for extraction of the organosulfur by-product. The hexane phase was decanted off and the extraction procedure repeated. The crude oil of 3-trifluoroacetamidopropyl 2-acetamido-3-O-acetyl-6-O-benzyl-2-deoxy-4-O-[2,3,4,6-tetra-O-acetyl-α/β-D-galactopyranosyl]-β-D-glucopyranoside (α/β-Ac4Gal-β-6-O-Bn-AcGlcNAc-sp3) (10) was dried in vacuo and used without additional purification.

The α/β-Ac4Gal-β-6-O-Bn-AcGlcNAc-sp3 (10) was dissolved in abs. methanol (2 L), treated with 2M sodium methoxide (NaOMe) (50 mL, 100 mmol) and the reaction mixture left for 4 h. The conversion was monitored by TLC (2:10:1 (v/v/v) isopropanol/ethyl acetate/water, Rf(α/β)˜0.25). The reaction mixture was neutralized with Dowex 50WX4 (50 g, H⁺-form) and stirred for 15 min. Activated charcoal (50 mL, granules) was added and the mixture was stirred for an additional 15 min before being filtered. The solution obtained was treated with pyridine (10 mL) and concentrated on a rotary evaporator, co-evaporated a few times with toluene, and dried in vacuo. The crude material was diluted with acetonitrile (1 L), stirred for 1 h at 40° C., cooled down and left in a fridge overnight for complete crystallization of the crude product. The crystalline material was collected on a glass filter, washed with cold acetonitrile and transferred to a 2 L beaker. Acetonitrile (1.5 L) was added, the suspension warmed up to boiling and some water (˜30 mL) added under reflux to complete dissolving of the crystals. The clear solution was cooled and left overnight in a fridge for complete crystallization. The crystalline product was collected on a glass filter, washed with cold acetonitrile, ethyl acetate and hexane, and dried under a fume hood overnight by vacuum drying. The total yield of 3-trifluoroacetamidopropyl 2-acetamido-6-O-benzyl-2-deoxy-4-O-[β-D-galactopyranosyl]-β-D-glucopyranoside (β-Gal-β-6-O-Bn-GlcNAc-sp3) (11) for last two steps: 40% (39 g, 63 mmol). ¹H NMR (CD₃OD): δ 1.75-1.88 (m, 2H, CCH₂C), 1.98 (s, 3H, NHC(O)CH₃), 3.23-3.30 (m, 1H, CH₂N), 3.25-3.30 (m, 1H, CH₂N), 3.40-3.46 (m, 2H, CH₂O, CH₂N), 3.49 (dd, J_{3,4} 1.1, J_{3,2} 6.3, H-3^{I}), 3.51-3.57 (m, 3H, H-5^{I}, H-5^{II}, H-2^{II}), 3.59-3.64 (m, 1H, H-3^{II}), 3.65-3.69 (m, 2H, H-4^{II}, H-6a^{I}), 3.71-3.77 (m, 2H, H-2^{I}, H-6b^{I}), 3.79 (dd, 1H, J_{4,3} 3.8, J_{4,5} 1.1, H-4^{I}), 3.85-3.93 (m, 3H, CH₂O, H-6a^{II}, H-6b^{II}), 4.31 (d, 1H, J_{1,2} 7.6, H-1^{I}), 4.39 (d, 1H, J_{1,2} 8.6, H-1^{II}), 4.50 (d, 1H, J 11.6, CH₂Ph), 4.62 (d, 1H, J 11.8, CH₂Ph), 7.24-7.41 (m, 5H, ArH); ¹³C NMR (CD₃OD): δ 22.9 (NHC(O)CH₃), 29.9 (CCH₂C), 38.1 (CH₂N), 56.7 (C-2^{I}), 62.5 (C-6^{I}), 67.9 (C-2^{II}), 69.8 (C-6^{II}), 70.3 (C-4^{I}), 74.1 (C-3^{I}), 74.4 (CH₂Ph), 74.8 (CH₂O), 77.1 (C-3^{II}), 72.5, 75.8 (C-5^{I}, C-5^{II}), 80.6 (C-4^{II}), 102.8 (C-1^{II}), 105.1 (C-1^{I}), 128.7, 128.9, 129.4 (C—Ar), 137.7 (NHC(O)CF₃), 173.6 (NHC(O)CH₃).

A major part of the β-Gal-β-6-O-Bn-GlcNAc-sp3 (11) (32 g, 51 mmol) was suspended in 2-dimethoxypropane (500 mL), treated with camphorsulfonic acid (2.4 g, 12.6 mmol) and stirred for a few days at 40° C. to complete the dissolution of the starting material. The reaction mixture was neutralized with pyridine (10 mL), concentrated on a rotary evaporator, and the residue co-evaporated with mixture of 1:1 (v/v) abs. methanol/toluene (100 mL). The oil obtained was dissolved in abs. methanol (500 mL) and stirred at 50° C. for 1 h. The conversion was monitored by TLC (9:3:2 (v/v/v) chloroform/ethyl acetate/methanol, Rf˜0.22). The solution was concentrated on a rotary evaporator, co-evaporated with ethyl acetate and dried in vacuo. The residual product was mixed with pyridine (200 mL) and Ac₂O (100 mL), and left for 48 h. Acetylation was monitored by TLC (10:1 (v/v) chloroform/methanol, Rf˜0.42). The reaction mixture was concentrated on a rotary evaporator, co-evaporated with toluene a few times, dried in vacuo. The crude product was diluted with chloroform (1 L), washed with water (500 mL), cold aq. saturated NaHCO₃ (500 mL), and then dried over anhydrous Na₂SO₄ (75 mL)/SiO₂ (25 mL). The solution was concentrated in vacuo (not to dryness), diluted with diethyl ether (1 L), and the product precipitated by addition of hexane (1 L, dropwise) with intensive stirring. For complete crystallization the mixture was left overnight. The crystalline product was collected on a glass filter, washed with a mixture of 4:1 (v/v) hexane/Et₂O followed by hexane and dried in vacuo. A crème-white dusty powder of 3-trifluoroacetamidopropyl 2-acetamido-6-O-benzyl-2-deoxy-4-O-[2,6-di-O-acetyl-3,4-O-isopropylidene-β-D-galactopyranosyl]-β-D-glucopyranoside (β-2′,6′-O-Ac-3′,4′-O-iPdGal-β-6-O-Bn-3-O-AcGlcNAc-sp3) (12) (34.5 g, 43.5 mmol) was obtained. Yield: 85%.

The powder of β-2′,6′-O-Ac-3′,4′-O-iPdGal-β-6-O-Bn-3-O-AcGlcNAc-sp3 (12) was dissolved in chloroform (1 L), then water (10 mL) was added under intensive stirring. The resulting emulsion was treated with TFA (100 mL) and stirred for an additional 2 h at r.t. (water bath). The conversion was monitored by TLC (10:1 (v/v) chloroform/methanol, Rf˜0.10). The reaction mixture was slowly poured into a 5 L beaker containing aq. saturated NaHCO₃ (3 L) under intensive mechanical stirring. The organic layer was separated, the aqueous layer was extracted with ethyl acetate (4×300 mL), and the combined organic layers dried over anhydrous Na₂SO₄. The solution was concentrated on a rotary evaporator, co-evaporated with toluene (not to dryness), and precipitated with hexane (300 mL). The resulting solid product was collected on a glass filter, washed with hexane, and dried in vacuo. Crude 3-trifluoroacetamidopropyl 2-acetamido-3-O-acetyl-6-O-benzyl-2-deoxy-4-O-[2,6-di-O-acetyl-β-D-galactopyranosyl]-β-D-glucopyranoside (β-2′,6′-O-AcGal-β-6-O-Bn-3-O-AcGlcNAc-sp3) (13) was used without additional purification.

The crude β-2′,6′-O-AcGal-β-6-O-Bn-3-O-AcGlcNAc-sp3 (13) was dissolved in dry acetonitrile (1 L), and triethyl orthoacetate (MeC(OEt)₃) (26 mL, 140 mmol) and p-toluene sulfonic acid (TsOH·H₂O) (2 g, 10 mmol) added. The reaction mixture was stirred for 3 h. The conversion was monitored by TLC (10:1 (v/v) chloroform/methanol Rf˜0.50). The mixture was neutralized with pyridine (20 mL), concentrated on a rotary evaporator, co-evaporated with toluene, and dried in vacuo.

The residue was dissolved in 80% aq. acetic acid (1 L) and stirred for 1.5 h. The conversion was monitored by TLC (10:1 (v/v) chloroform/methanol, Rf˜0.32). The resulting solution was concentrated on a rotary evaporator, co-evaporated with toluene, dried in vacuo.

The residue was dissolved in ethyl acetate (300 mL), filtered through a thin layer of SiO₂, and washed with ethyl acetate (100 mL). To the resulting solution was added hexane under stirring until light turbidity appeared. The mixture was allowed to crystallize for 1 h under stirring. An additional portion of hexane (total volume of hexane added to the ethyl acetate solution was 800 mL) was added dropwise under stirring, and the mixture left overnight for complete crystallization of the product. The crude crystalline material was collected on a glass filter, washed with 4:1 (v/v) hexane/ethyl acetate, and then pure hexane. [If the crude material contained admixtures that hindered crystallization, filtration through SiO₂ on a glass filter (50 g per 1 g of crude product) eluting with pure ethyl acetate was substituted for the first crystallisation. The main fraction collected from 70 mL to 250 mL. TLC in 15:1 (v/v) ethyl acetate/isopropanol (Rf˜0.44).] The crystallization procedure was repeated once more with the same amount of solvents. After drying in vacuo the target β-2′,4′,6′-O-AcGal-β-6-O-Bn-3-O-AcGlcNAc-sp3 (14) was provided as a white powder (18 g, 23 mmol). Total yield for last two steps: 53%. ¹H NMR (CDCl₃): δ 1.74-1.84 (m, 2H, CCH₂C), 1.96, 2.08, 2.14 (3s, 12H, 4C(O)CH₃), 2.04 (s, 3H, NHC(O)CH₃) 3.21-3.30 (m, 1H, CH₂N), 3.49 (ddd, 1H, J_{5,6a}˜J_{5,6b}˜2.2, J_{5,4} 9.8, H-5^{I}), 3.54-3.66 (2m, 4H, H-5^{I}, H-3^{I}, CH₂O, CH₂N), 3.73 (dd, 1H, J_{6a,6b} 10.8, J_{6a,5} 2.1, H-6a^{I}), 3.78 (dd, 1H, J_{6b,6a} 10.8, J_{6b,5} 2.1, H-6b^{I}), 3.89-3.97 (m, 2H, H-4^{I}, CH₂O), 3.99-4.06 (m, 2H, H-2^{I}, H-6b^{I}), 4.10 (dd, 1H, J_{6a,6b} 11.3, J_{6a,5} 6.7, H-6a^{II}), 4.40 (d, 1H, J_{1,2} 3.8, H-1^{II}), 4.41 (d, 1H, J_{1,2} 4.1, H-1^{I}), 4.48 (d, 1H, J 12.0 CH₂Ph), 4.72 (d, 1H, J 12.0 CH₂Ph), 4.78 (dd, 1H, J_{2,3} 7.9, J_{2,1} 2.2, H-2^{II}), 5.01 (dd, 1H, J_{3,4} 9.1, J_{3,2} 1.1, H-3^{I}), 5.23 (dd, 1H, J_{4,5} 1,3, J_{4,3} 3.8, H-4^{II}), 5.93 (d, 1H, J_{NH,2} 8.9, NHAc), 7.30-7.41 (m, 5H, ArH), 7.51 (br t, 1H, NH(O)CF₃); ¹³C NMR (CDCl₃): δ 23.4 (NHC(O)CH₃), 28.6 (CCH₂C), 20.7, 20.8, 20.9, 21.1 (4C(O)CH₃), 37.5 (CH₂N), 53.8 (C-2^{I}), 61.6 (C-6^{II}), 66.7 (CH₂O), 67.8 (C-6^{I}), 69.6 (C-4^{II}), 72.8 (C-3^{I}), 72.9 (C-2^{II}), 70.9, 71.4 (C-5^{II}, C-3^{II}), 73.1 (C-2^{I}), 73.8 (CH₂Ph), 74.8 (C-4^{I}), 75.1 (C-5^{I}), 100.3 (C-1^{II}), 101.7 (C-1^{I}), 128.1, 128.2, 128.7 (C—Ar), 137.9 (NHC(O)CF₃), 170.6-171.4 (4C(O)CH₃).

### Preparation of Co-Aminoalkyl Trisaccharides 3-aminopropyl 2-acetamido-2-deoxy-4-O-[(3-O-α-D-galactopyranosyl)-β-D-galactopryanosyl]-β-D-glucopyranoside (Galα3Galβ4GlcNAc-(CH₂)₃NH₂) (Galili-(CH₂)₃NH₂) (18)

Target Galα3Galβ4GlcNAc-(CH₂)₃NH₂ (18) was prepared according to SCHEME III as disclosed in the publication of Kunetskiy et al (2020). β-Ac₄GalSEt (9) (60.5 g, 154 mmol) was dissolved in abs. methanol (500 mL), then 2M NaOMe (5 mL, 10 mmol) was added, and the reaction left for 12 h. The conversion was monitored by TLC (3:1 (v/v) ethanol/chloroform, Rf˜0.35). The mixture was neutralised by Dowex50WX4 (5 g, H⁺-form) and filtered after 15 min of stirring. The solution was concentrated on a rotary evaporator, co-evaporated with abs. dimethylformamide (50 mL) and dried in vacuo.

The residual oil was dissolved in abs. dimethylformamide (500 mL), the solution was cooled to 0° C. and NaH (32 g, 55% in mineral oil, 0.75 mol) added under intensive stirring. Benzyl bromide (BnBr) (100 mL, 0.85 mol) was added dropwise, the mixture stirred for 24 h, and then left without stirring for an additional 24 h. The conversion was monitored by TLC (4:1 (v/v) hexane/ethyl acetate, Rf˜0.48). Methanol (30 mL) was added under intensive stirring, the mixture poured into water (1 L), and extracted with toluene (3×0.5 L). The combined organic layers were washed with water (0.5 L) and dried with Na₂SO₄. The solution was concentrated on a rotary evaporator and dried in vacuo. The crude product was diluted with diethyl ether (120 mL) and the solution obtained added dropwise to hexane (600 mL) under stirring at 0° C. The crystalline product was precipitated once again under the same conditions, collected on a glass filter, washed with a portion of hexane, and dried in vacuo to provide 1-thioethyl-2,3,4,6-tetra-O-benzyl-β-D-galactopyranoside (β-

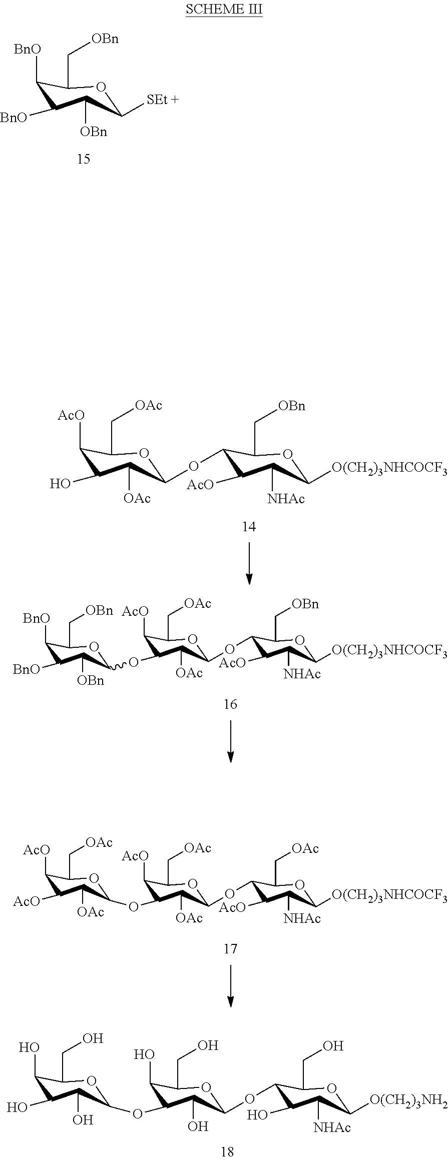

Bn₄GalSEt) (15) (53 g, 91 mmol). Yield: 60%. NMR spectra were in accordance with those disclosed in the publication of Weïwer et al (2008).

To a solution of β-Bn₄GalSEt (15) (21.0 g, 36 mmol) in abs. dichloromethane (200 mL) was added bromine (2 mL, 39.6 mmol) at 0° C. The reaction was left at 0° C. for 1 h. The solvent was evaporated and the residue co-evaporated with toluene (2×100 mL) to remove traces of bromine. The almost colourless oil of 2,3,4,6-tetra-O-benzyl-β-D-galactopyranosyl bromide (Bn₄GalBr) was dried in vacuo.

To a solution of β-2′,4′,6′-O-AcGal-β-6-O-Bn-3-O-AcGlcNAc-sp3 (14) (18.0 g, 24 mmol) in abs. dichloromethane (240 mL) were added freshly dried MS-4 Å (30 mL) and tetramethylurea (5.2 mL, 43.2 mmol). The mixture was stirred for 30 min, then AgOTf (10.2 g, 39.6 mmol) was added and the mixture stirred for an additional 15 min. A solution of Bn₄GalBr in abs. dichloromethane (120 mL) was added to the mixture dropwise under intensive stirring. The reaction was left for 24 h with stirring. The conversion was monitored by TLC (10:1 (v/v) chloroform/methanol, Rf˜0.40). The reaction mixture was treated with pyridine (20 mL), filtered through Celite on a glass filter, and solid material washed with a portion of dichloromethane and a mixture of 10:1 (v/v) dichloromethane/methanol.

The filtrate was poured into a 10% aq. solution of Na₂S₂O₃ (250 mL), washed with cold aq. saturated NaHCO₃ (200 mL), and dried with Na₂SO₄/SiO₂. The resulting solution was concentrated on a rotary evaporator, co-evaporated with toluene, and dried in vacuo. The product 3-trifluoroacetamidopropyl 2-acetamido-3-O-acetyl-6-benzyl-4-O-(2,4,6-tri-O-acetyl-3-O-[2,3,4,6-tetra-O-benzyl-α/β-D-galactopyranosyl]-β-D-galactopyranosyl)-2-deoxy-β-D-glucopyranoside (16) used at the next stage without additional purification.

The product (16) was dissolved in methanol (1 L), then 10% Pd/C (10 g) was added, and the reaction mixture degassed further in vacuo by filling with H₂ (1 atm) three times. The reaction was left under H₂ (1 atm) with stirring for 48 h. The conversion was monitored by TLC (2:10:1 (v/v/v) isopropanol/ethyl acetate/water, Rf˜0.21). The reaction mixture was filtered through Celite on a glass filter and the solid material washed with methanol. The resulting solution was concentrated on a rotary evaporator, co-evaporated with acetonitrile, and dried in vacuo. The residue was dissolved in pyridine (100 mL) and Ac₂O (50 mL) and the reaction mixture was left for 48 h. The acetylation was monitored by TLC (10:1 (v/v) chloroform/methanol, Rf˜0.30). The reaction mixture was concentrated on a rotary evaporator, co-evaporated with toluene, diluted with chloroform (1 L), washed with cold aq. saturated NaHCO₃ (500 mL), and dried with Na₂SO₄/SiO₂. The resulting solution was concentrated on a rotary evaporator, dried in vacuo to solid foam. The raw product was dissolved in ethyl acetate (600 mL) and hexane added to the resulting solution under stirring until light turbidity appeared. The mixture was allowed to crystallize for 12 h. Crystalline material was collected on a glass filter, washed with 3:1 (v/v) hexane/ethyl acetate followed by pure hexane, and dried in vacuo to provide a white powder of 3-trifluoroacetamidopropyl 2-acetamido-3,6-di-O-acetyl-4-O-(2,4,6-tri-O-acetyl-3-O-[2,3,4,6-tetra-O-acetyl-α-D-galactopyranosyl]-β-D-galactopyranosyl)-2-deoxy-β-D-glucopyranoside (Ac₉Galα/β3Galβ4GlcNAc-sp3) (17) (13 g, 12 mmol) Yield: 50%. NMR spectra were in accordance with those disclosed in the publication of Pazynina et al (2002).

Ac₉Galα/β3Galβ4GlcNAc-sp3 (17) (13 g, 12 mmol) was dissolved in abs. methanol (600 mL), then 2M NaOMe (30 mL, 60 mmol) was added, and the reaction left for 1 h. The reaction mixture was concentrated on a rotary evaporator (not to dryness) and water (600 mL) was then added. The mixture was left for an

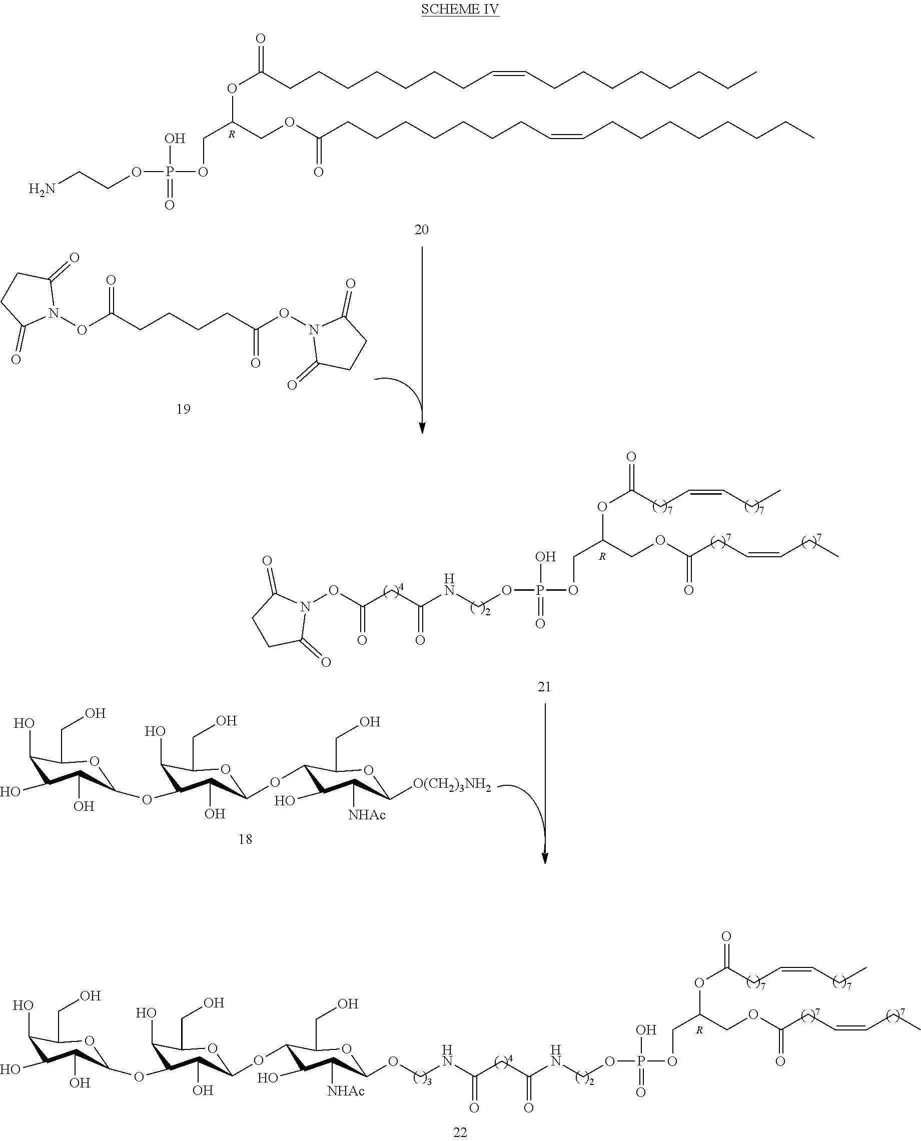

additional 12 h before transfer to a glass filter with Dowex50WX4 (300 mL for capacity 1.1 meq/mL, H⁺-form). The resin was washed with double distilled water (1 L), 1M aq. pyridine (1 L) and double distilled water (200 mL). The target Galα3Galβ4GlcNAc-(CH₂)₃NH₂ (18) was washed from the resin with 1 M aq. NH₃ (1.5 L) into a separate flask. (TLC control in 5:1 (v/v) MeOH/1M aq. pyridinium acetate Rf˜0.38). The solution was concentrated on a rotary evaporator and twice freeze dried with a little double distilled water to provide the target Galα3Galβ4GlcNAc-(CH₂)₃NH₂ (18) as a white solid (7.45 g, 12.4 mmol). Yield: 95%. NMR spectra were in accordance with those disclosed in the publication of Pazynina et al (2002).

### Preparation of 1,2-dioleoylphosphatidylethanolamine (DOPE) Conjugate of Galα3Galβ4GlcNAc (Galili) (22)

The target glycoconjugate (22) was prepared according to SCHEME IV as disclosed in the publication of Bovin et al (2016).

To a solution of bis(N-hydroxysuccinimidyl) adipate (19) (70 mg, 205 mol) in dry N,N-dimethylformamide (1.5 ml), 1,2-O-dioleoyl-sn-glycero-3-phosphatidylethanolamine (20) (40 mol) in chloroform (1.5 ml) is added, followed by triethylamine (7 μl). The mixture is kept for 2 h at room temperature, then neutralized with acetic acid and partially concentrated under vacuum. Column chromatography (Sephadex LH-20, 1:1 (v/v) chloroform/methanol, 0.2% acetic acid) of the residue yields the activated lipid (21) (37 mg, 95%) as a colorless syrup.

¹H NMR (CDCl₃/CD₃OD, 2:1) 5.5 (m, 4H, 2×(—CH═CH—), 5.39 (m, 1H, —OCH₂—CHO—CH₂O—), 4.58 (dd, 1H, J=3.67, J=11.98, —CCOOHCH—CHO—CH₂O—), 4.34 (dd, 1H, J=6.61, J=11.98, —CCOOHCH—CHO—CH₂O—), 4.26 (m, 2H, PO—CH₂—CH₂—NH₂), 4.18 (m, 2H, —CH₂—OP), 3.62 (m, 2H, PO—CH₂—CH₂—NH₂), 3.00 (s, 4H, ONSuc), 2.8 (m, 2H, —CH₂—CO (Ad), 2.50 (m, 4H, 2×(—CH₂—CO), 2.42 (m, 2H, —CH₂—CO (Ad), 2.17 (m, 8H, 2×(—CH₂—CH═CH—CH₂—), 1.93 (m, 4H, COCH₂CH₂CH₂CH₂CO), 1.78 (m, 4H, 2×(COCH₂CH₂—), 1.43, 1.47 (2 bs, 40H, 20CH₂), 1.04 (m, 6H, 2CH₃). R_{f} 0.5 (chloroform-methanol-water, 6:3:0.5.

To a solution of the activated lipid (21) (33 mol) in N,N-dimethylformamide (1 ml), 30 μmol of Galα3Galβ4GlcNAc-(CH₂)₃NH₂ (18) and 5 μl of triethylamine (Et₃N) is added. The mixture is stirred for 2 h at room temperature. Column chromatography on silica gel (6:5:1 (v/v/v) dichloromethane/ethanol/water) provides an 84% yield of the glycoconjugate AGI-134 (22).

AGI-134 (22): ¹H NMR (700 MHz, CDCl₃-CD₃OD, 1:1 v/v, selected signals), δ, ppm: 1.05 (t, 6H, J 6.98, 2CH₃), 1.36-1.55 (m, 40H, 20CH₂), 1.73-1.84 (m, 8H, COCH₂CH₂CH₂CH₂CO and 2×(COCH₂CH₂—), 1.85-1.96 (m, 2H, O—CH₂CH₂CH₂—NH), 2.14-2.22 (m, 11H, 2×(—CH₂—CH═CH—CH₂—), NHC(O)CH₃), 2.45-2.52 (m, 4H, 2×—CH₂—CO), 2.36-2.45 (m, 4H, 2×—CH₂—CO), 3.29-3.35 (m, 1H, —CH₂—CHH—NH), 3.52-3.62 (m, 3H, PO—CH₂—CH₂—NH, —CH₂—CHH—NH), 4.13-4.18 (m, 2H, —CHO—CH₂OP—), 4.19 (d, 1H, J_{3,4} 2.48, H-4^{II}), 4.36 (dd, 1H, J 6.8, J 12.00, —C(O)OCHHCHOCH₂O—), 4.56 (d, 1H, J_{1,2} 8.39, H-1^{I}), 4.60 (dd, 1H, J 2.87, J 12.00, C(O)OCHHCHOCH₂O—), 4.61 (d, 1H, J_{1,2} 7.57, H-1^{II}), 5.18 (d, 1H, J_{1,2} 2.52, H-1^{III}), 5.34-5.43 (m, 1H, —OCH₂—CHO—CH₂O—), 5.45-5.54 (m, 4H, 2×—CH═CH—). R_{f} 0.45 (CH₂Cl₂-EtOH—H₂O; 6:5:1).

Although the invention has been described with reference to embodiments or examples it should be appreciated that variations and modifications may be made to these embodiments or examples without departing from the scope of the invention. Where known equivalents exist to specified elements, features, integers, or other limitations, of the matter described in the Summary of Invention, such equivalents are incorporated as if specifically referred to in this specification. For example, it is anticipated that one skilled in the art could readily identify and confirm the suitability for use of alternatives to the organic solvents specified in the embodiments. Variations and modifications to the embodiments or examples that include elements, features, integers, or other limitations, disclosed in and selected from the referenced publications are within the scope of the invention unless specifically disclaimed. The advantages provided by the invention and discussed in the description included in this specification may be provided in the alternative or in combination in these different embodiments of the invention.

### INDUSTRIAL APPLICABILITY

Scalable methods for the preparation of disaccharide derivatives for use in the preparation of glycoconjugates comprising immunologically significant tri-, tetra- and oligosaccharides are described.

### INCORPORATION BY REFERENCE

For the purposes of 37 C.F.R. 1.57 of the United States Code of Federal Regulations the disclosures of the following publication(s) (as more specifically identified under the heading ‘Referenced Publications’) are incorporated by reference: Kunetskiy et al (2020); Romano and Oscarson (2019).

### REFERENCED PUBLICATIONS

Bobkov et al (2008) Phosphoramidite building blocks for efficient incorporation of 2′-O-aminoethoxy(and propoxy)methyl nucleosides into oligonucleotides Tetrahedron 64, 6238-6251.
Boren et al (1973) Benzylated orthoesters in glycoside synthesis Acta Chem. Scand., 27, 2639-2644.
Bovin et al (1988) Artificial antigens and affinity sorbents with groups specificities Le^{a}, Le^{b} and Le^{d} Khimiya Prirodynkh Soedinenii, 6, 777-785.
Bovin et al (2016) Multivalent ligand lipid constructs international application no. PCT/NZ2015/050197 [publ. no. WO 2016/080850 A1].
Chinarev et al (2021) Synthesis of spacer armed Kdn(2->6′) and (2->3′)-lactosamines for immunochemical research Mendeleev Commun., 31, 490-492.
Gagarinov et al (2019) Protecting-group-controlled enzymatic glycosylation of oligo-N-acetyllactosamine derivatives Angew. Chem. Int. Ed. 58, 10547-10552.

Hanessian et al (2001a) Practical syntheses of B disaccharide and linear B type 2 trisaccharide—non-primate epitope markers recognized by human anti-α-Gal antibodies causing hyperacute rejection of xenotransplants Tetrahedron 57, 3267-3280.
Hindsgaul et al (1982) Synthesis of type 2 human blood-group antigenic determinants. The H, X, and Y haptens and variations of the H type 2 determinant as probes for the combining site of the lectin I of Ulex europaeus Carbohydrate Research, 109, 109-142.
Kunetskiy et al (2020) Synthesis of blood group A and B (type 2) tetrasaccharides. A strategy with fucosylation at the last stage Carbohydrate Research 498, 108192.
Lemieux et al (1979) Glycoside-ether-ester compounds U.S. Pat. No. 4,137,401.
Lönn (1984) Dissertation, University of Stockholm; Abstr. XII Th Int. Carbohydr. Symp. 88, 116.
Mukhopadhyay and Field (2003) A simple one-pot method for the synthesis of partially protected mono- and disaccharide building blocks using an orthoesterification-benzylation-orthoester rearrangement approach Carbohydrate Research 338, 2149-2152.
Paulsen (1985) Strategies in oligosaccharide synthesis Organic Synthesis An Interdisciplinary Challenge, Proceedings of the 5^{th} IUPAC Symposium on Organic Synthesis, eds. Streith, Prinzbach and Schill, Blackwell Scientific Publications.
Pazynina et al (2002) Synthesis of histo blood-group antigens A and B (type 2), xenoantigen Galα1-3Galβ1-4GlcNAc and related type 2 backbone oligosaccharides as haptens in spacered form Mendeleev Commun. 12(4), 143-145.
Pazynina et al (2008) The synthesis of linear trilactosamine Russian Journal of Bioorganic Chemistry 34, No. 5, 625-631.
Pazynina et al (2010) Koenigs-Knorr glycosylation with neuraminic acid derivatives International Journal of Carbohydrate Chemistry Volume 2010, Article ID 594247, 8 pages doi:10.1155/2010/594247.
Romano and Oscarson (2019) Synthesis of lactosamine-based building blocks on a practical scale and investigations of their assembly for the preparation of ¹⁹F-labelled LacNAc oligomers Org. Biomol. Chem., 17, 2265-2278.
Ryzhov et al (2019) Synthesis of N-acetyllactosamine based branched hexasaccharide Mendeleev Commun. 29, 680-682.
Severov et al (2007) Synthesis of N-acetyllactosamine-containing oligosaccharides, galectin ligands Russian Journal of Bioorganic Chemistry, Vol. 33, No. 1, 122-138.
Weiver et al (2008) Synthesis of floridoside Journal of Carbohydrate Chemistry, 27, 420-427.
Zinin et al (2007) Use of methanesulfonic acid in the reductive ring-opening of O-benzylidene acetals Carbohydrate Research 342, 627-630.

1) An N-acetyllactosamine derivative of the structure: 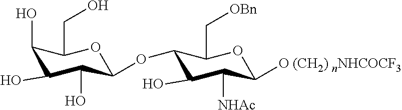
where n is the integer 2, 3, 4, 5, 6, 7 or 8.

2) The N-acetyllactosamine derivative of claim 1 where the N-acetyllactosamine derivative is in a crystalline form.

3) The N-acetyllactosamine derivative of claim 1 or 2 where n is the integer 2, 3 or 4.

4) The N-acetyllactosamine derivative of claim 3 where n is the integer 3.

5) A batch quantity of the N-acetyllactosamine derivative of any one of claims 1 to 4 where the quantity is greater than 10 q.

6) A method for the batch preparation of a quantity of the N-acetyllactosamine derivative of any one of claims 1 to 4 comprising:
deacetylating an anomeric mixture of a ω-trifluoroacetamidoalkyl disaccharide derivative of the structure: 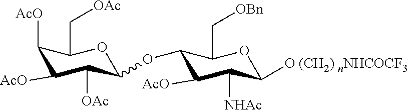
to provide a reaction product;
preparing a solution of the reaction product in a solvent; and then
precipitating the quantity of the N-acetyllactosamine derivative from the solution, where the quantity is greater than 10 g.

7) The method of claim 6 where the solvent is at least 98% (v/v) acetonitrile.

8) Use of a quantity of the N-acetyllactosamine derivative of any one of claims 1 to 4 in the preparation of an ω-aminoalkyl glycoside of the structure: 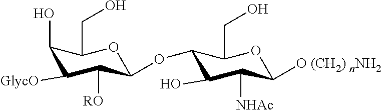
where R is H or Glyc, and Glyc is a glycan.

9) The use of claim 8 where the quantity is greater than 10 g.

10) The use of claim 9 where Glyc is a monosaccharide residue.

11) The use of claim 10 where R is H.

12) The use of claim 11 where Glyc is α-D-galactopyranosyl.
